SEQUENCE LISTING (23 sequences)

SEQ ID NO: 1 - DNA, 229 bp, Artificial Sequence
  Amplified region of the N gene of SARS-CoV-2
aacacaagctttcggcagacgtggtccagaacaaacccaaggaaattttggggaccaggaactaatcagacaaggaactgattacaaacattggccgcaaattgcacaatttgcccccagcgcttcagcgttcttcggaatgtcgcgcattggcatggaagtcacaccttcgggaacgtggttgacctacacaggtgccatcaaattggatgacaaagatccaaatttc

SEQ ID NO: 2 - DNA, 18 bp, Artificial Sequence
  N gene-F3
aacacaagctttcggcag

SEQ ID NO: 3 - DNA, 22 bp, Artificial Sequence
  N gene-B3
gaaatttggatctttgtcatcc

SEQ ID NO: 4 - DNA, 41 bp, Artificial Sequence
  N gene-FIP
tgcggccaatgtttgtaatcagccaaggaaattttggggac

SEQ ID NO: 5 - DNA, 39 bp, Artificial Sequence
  N gene-BIP
cgcattggcatggaagtcactttgatggcacctgtgtag

SEQ ID NO: 6 - DNA, 19 bp, Artificial Sequence
  N gene-LB
ttccttgtctgattagttc

SEQ ID NO: 7 - DNA, 18 bp, Artificial Sequence
  N gene-LF
accttcgggaacgtggtt

SEQ ID NO: 8 - DNA, 234 bp, Artificial Sequence
  Amplified region of the E gene of SARS-CoV-2
ccgacgacgactactagcgtgcctttgtaagcacaagctgatgagtacgaacttatgtactcattcgtttcggaagagacaggtacgttaatagttaatagcgtacttctttttcttgctttcgtggtattcttgctagttacactagccatccttactgcgcttcgattgtgtgcgtactgctgcaatattgttaacgtgagtcttgtaaaaccttctttttacgtttactct

SEQ ID NO: 9 - DNA, 18 bp, Artificial Sequence
  E gene-F3
ccgacgacgactactagc

SEQ ID NO: 10 - DNA, 23 bp, Artificial Sequence
  E gene-B3
agagtaaacgtaaaaagaaggtt

SEQ ID NO: 11 - DNA, 41 bp, Artificial Sequence
  E gene-FIP
acctgtctcttccgaaacgaatttgtaagcacaagctgatg

SEQ ID NO: 12 - DNA, 41 bp, Artificial Sequence
  E gene-BIP
ctagccatccttactgcgctactcacgttaacaatattgca

SEQ ID NO: 13 - DNA, 19 bp, Artificial Sequence
  E gene-LB
tgagtacataagttcgtac

SEQ ID NO: 14 - DNA, 19 bp, Artificial Sequence
  E gene-LF
tcgattgtgtgcgtactgc

SEQ ID NO: 15 - DNA, 46 bp, Artificial Sequence
  RT-LAMP amplicon of the N gene
aattgcacaatttgcccccagcgcttcagcgttcttcggaatgtcg

SEQ ID NO: 16 - RNA, 41 nt, Artificial Sequence
  gRNA for the N gene
uaauuucuacuaaguguagaucccccagcgcuucagcguuc

SEQ ID NO: 17 - DNA, 50 bp, Artificial Sequence
  Activator for the N gene
gcaaattgcacaatttgcccccagcgcttcagcgttcttcggaatgtcgc

SEQ ID NO: 18 - DNA, 60 bp, Artificial Sequence
  RT-LAMP amplicon of the E gene
acgttaatagttaatagcgtacttctttttcttgctttcgtggtattcttgctagttaca

SEQ ID NO: 19 - RNA, 41 nt, Artificial Sequence
  gRNA for the E gene
uaauuucuacuaaguguagaugugguauucuugcuaguuac

SEQ ID NO: 20 - DNA, 67 bp, Artificial Sequence
  Amplified fragments of SARS-CoV-2 (N2)
ttacaaacattggccgcaaattgcacaatttgcccccagcgcttcagcgttcttcggaatgtcgcgc

SEQ ID NO: 21 - DNA, 20 bp, Artificial Sequence
  N2-Forward primer
ttacaaacattggccgcaaa

SEQ ID NO: 22 - DNA, 18 bp, Artificial Sequence
  N2-Reverse primer
gcgcgacattccgaagaa

SEQ ID NO: 23 - DNA, 23 bp, Artificial Sequence
  N2-Probe
  6-FAM (1)..(1)
  BHQ_1 (23)..(23)
acaatttgcccccagcgcttcag

(19) United States
(12) Patent Application Publication
(10) Pub. No.: US 2022/0136074 A1
(43) Pub. Date: May 5, 2022

(54) ISOTHERMAL AMPLIFICATION AND AMBIENT VISUALIZATION IN A SINGLE TUBE FOR THE DETECTION OF SARS-COV-2 USING LOOP-MEDIATED AMPLIFICATION AND CRISPR TECHNOLOGY

(71) Applicant: The Governors of the University of Alberta, Edmonton (CA)

(72) Inventors: Bo PANG, Edmonton (CA); Hongquan ZHANG, Ottawa (CA); David Lorne TYRRELL, Edmonton (CA); Xiaochun Chris LE, Edmonton (CA)

(21) Appl. No.: 17/519,670

(22) Filed: Nov. 5, 2021

Related U.S. Application Data
(60) Provisional application No. 63/110,091, filed on Nov. 5, 2020.

(51) Int. Cl. C12Q 1/70; C12N 9/22; C12Q 1/6844

(52) U.S. Cl. CPC C12Q 1/701; C12Q 1/6844; C12N 9/22

(57) ABSTRACT

Isothermal amplification and ambient visualization in a single tube for the detection of SARS-CoV-2 using loop-mediated amplification and CRISPR technology.

### SEQUENCE LISTING

This application contains a Sequence Listing which has been filed electronically in ASCII format and is hereby incorporated by reference in its entirety. Said ASCII copy, created on Nov. 4, 2021, is named 51012-043002_Sequence_Listing_11_4_21_ST25.txt and is 5,392 bytes in size.

### FIELD

The present disclosure relates generally to Isothermal amplification and ambient visualization in a single tube for the detection of SARS-CoV-2 using loop-mediated amplification and CRISPR technology.

### BACKGROUND

Effective control, containment, and mitigation of the coronavirus disease 2019 (COVID-19) pandemic require accessible tools and assays for timely diagnosis of the disease. The widely used molecular diagnostic assay relies on reverse transcription (RT) polymerase chain reaction (PCR), which enables the detection of specific genes in SARS-CoV-2, the virus that causes COVID-19. Specific sequences of any of the four genes that encode the RNA-dependent RNA polymerase (RdRp), envelope (E), nucleocapsid (N), and spike (S) proteins of SARS-CoV-2 are reverse transcribed and exponentially amplified in diagnostic tests. The exponential amplification during PCR enables detection of a few copies (molecules) of the gene sequence, and results in ultrasensitive detection of SARS-CoV-2 in infected individuals.

Global demands and competition for COVID-19 test kits and reagents necessitate the development of alternative assays and diagnostic tools. In addition, the need for thermal cycling for PCR tests makes point-of-care testing and on-site analysis in remote communities challenging. Consequently, there has been much interest in the development of molecular assays using isothermal amplification of nucleic acids.¹ Two isothermal amplification techniques, loop-mediated isothermal amplification (LAMP) and recombinase polymerase amplification (RPA), have been demonstrated to achieve the detection of SARS-CoV-2 RNA. LAMP is particularly promising for point-of-care applications because it requires only a single enzyme for the exponential amplification.²

Recent advances in clustered regularly interspaced short palindromic repeats (CRISPR) and CRISPR-associated (Cas) proteins have stimulated the application of CRISPR technology and isothermal amplification techniques to the development of diagnostic assays for COVID-19. Broughton et al.³ combined RT-LAMP with CRISPR-Cas12a and developed a CRISPR-based assay for the detection of SARS-CoV-2. Named SARS-CoV-2 DNA endonuclease-targeted CRISPR trans reporter (DETECTR), this assay reduced the analysis time to <40 min, eliminated thermal cycling, and produced a color readout visible on lateral flow strips. The assay is potentially useful for point-of-care applications, although it required three steps: RT-LAMP amplification, CRISPR-mediated cleavage of reporters, and lateral flow display of results. The assay is conducted in two separate tubes under three temperature conditions: 62° C., 37° C., and room temperature. The use of two controlled temperatures (62° C. and 37° C.) increases the device requirement for the assay. Opening tubes to transfer reaction reagents and products increases the risk of potential contamination, which compromises the applicability of the assay for point-of-care testing.

### SUMMARY

The present disclosure relates generally to Isothermal amplification and ambient visualization in a single tube for the detection of SARS-CoV-2 using loop-mediated amplification and CRISPR technology.

In one aspect there is provided a method of detecting SARS-CoV-2 in a sample from a subject having a SARS-CoV-2 infection, or suspected of having a SARS-CoV-2 infection, or at risk of a SARS-CoV-2 infection, comprising:

(a) subjecting the sample to reverse transcription loop-mediated exponential amplification (RT-LAMP) to amplify a target sequence with said SARS-CoV-2 and generate an RT-LAMP amplicon comprising the target sequence;

(b) contacting the RT-amplicon with a reaction mixture at a reaction temperature for a reaction time, the reaction mixture comprising:

a guide RNA (gRNA) comprising a polynucleotide sequence complementary to the target sequence recognizing,

a Cas12a protein; and

a ssDNA reporter molecules comprising a first end having a quencher molecule and a second end having a fluorophore

(c) detecting a signal indicative of amplification of the region of the target nucleic acid molecule,

wherein, step (a) and step (b) are performed sequentially in a single unopened container,

wherein, if a signal detected, the subject is determined to have a SARS CoV-2 infection,

wherein, if a signal is not detected, the subject is determined to not to have a SARS CoV-2 infection.

In one example, the target sequence is the E gene or N gene.

In one example, the gRNA comprises or consists of 41 nucleotides.

In one example, the gRNA comprises or consists of the nucleotide sequence of SEQ ID NO: 16 or SEQ ID NO: 19.

In one example, the ssDNA reporter comprises or consists of 8 nucleotides.

In one example, wherein the ssDNA reporter comprises or consists of the nucleotide sequence 6-FAM/TTATTATT/IABk.

In one example, wherein the quencher molecule is Iowa Black® fluorescence quencher and the fluorophore is 6-carboxyfluorescein.

In one example, said reverse transcription loop-mediated exponential amplification (RT-LAMP) is carried out at about 62° C. for about 30 min.

In one example, wherein the reaction time in step (b) is about 10 minutes and the reaction temperature is about 23° C.

In one example, the reagents for step (a) and step (b) are lyophilized and present in separate compartments of the single container, before addition of the sample.

In one example, said subject is a human.

In one aspect there is provided an isolated polynucleotide comprising or consisting of the nucleotide sequence of SEQ ID Nos. 1, 2, 3, 4, 5, 6, 7, 8, 9, 10, 11, 12, 13, 14,15,16,17,18,19,20,21,22, or 23.

In one aspect there is provided a kit comprising one or more isolated polynucleotides comprising or consisting of the nucleotide sequence of SEQ ID Nos. 1, 2, 3, 4, 5, 6, 7, 8, 9, 10, 11, 12, 13, 14, 15, 16, 17, 18, 19, 20, 21, 22, or 23, and optionally a container, and/or optionally instructions for the use thereof.

### BRIEF DESCRIPTION OF THE FIGURES

Embodiments of the present disclosure will now be described, by way of example only, with reference to the attached Figures.

FIGS. 1A and 1B. Schematics showing (FIG. 1A) the general principle and (FIG. 1B) the overall operation of the assay for the detection of SARS-CoV-2. (FIG. 1A) The specific gene sequence of SARS-CoV-2 RNA is amplified using RT-LAMP. The RT-LAMP products are scanned by the Cas12a-gRNA ribonucleoprotein (RNP) complex. The sequence in viral RNA marked in blue color is the protospacer adjacent motif (PAM), which is essential for Cas12a recognition. The RNP binds to the specific sequence (in orange color) complementary to gRNA, activating the trans-cleavage activity of Cas12a. The active Cas12a system cleaves a short ssDNA reporter (8 nt) that is labeled with a fluorophore and a quencher on either end. The cleavage of the reporter separates the quencher from the fluorophore, resulting in the generation of fluorescence. (FIG. 1B) A 0.2-mL PCR tube contains the RT-LAMP reagent mixture (25 μL or lyophilized) at the bottom and the Cas12a-mediated detection reagent mixture (10 μL liquid or lyophilized) inside the cap. An aliquot (2-10 μL) of the RNA extract, and 25 μL buffer if operated with lyophilized reagents, is added into the bottom of the tube, mixing with the RT-LAMP reagents. The tube is placed in a temperature controller and the bottom of the tube is maintained at 62° C. for 30 min to allow for RT-LAMP reactions. The tube is then removed from the temperature controller, and the subsequent procedures are performed at the room temperature. Inverting and wrist-flicking of the tube makes the Cas12a reagents mix with the RT-LAMP amplicons in the bottom. Green fluorescence is generated at the room temperature and is visualized under the excitation of a handheld UV lamp.

FIGS. 2A and 2B. Incorporating CRISPR Cas12a-mediated detection with RT-LAMP amplification improves the specificity. (FIG. 2A) RT-LAMP amplification curves from triplicate analyses of the target N gene and the negative controls, with the real-time fluorescence detection of intercalating SYBR Green. (FIG. 2B) CRISPR Cas12a-mediated detection of the RT-LAMP products. In this set of experiments, the 25 μL of RT-LAMP reaction solution contained 1×NEBuffer 2.1 buffer, 1.4 mM deoxynucleotide (dNTP), 0.2 μM each of the outer primers (F3 and B3), 1.6 μM each of the inner primers (FIP and BIP), 0.8 μM each of the loop primers (LF and LB), 4 units of RNase inhibitor, 7.5 units of WarmStart® RTx reverse transcriptase, 8 units of Bst 2.0 DNA polymerase, and 5 μL of 750 copies/μL viral RNA (as target) or nuclease-free water (as negative control). RT-LAMP reactions were performed at 62° C., and the reaction products were monitored using either SYBR Green (FIG. 2A) or the CRISPR Cas12a system (FIG. 2B).

FIG. 3. Fluorescence generated from the enzymatic cleavage of ssDNA reporters by Cas12a-gRNA maintained at different temperatures. Ten μL of 50 nM of Cas12a-gRNA (recognizing the N gene) was maintained at the specified temperature for 30 min. Fifteen μL of the mixture of DNA activator (for the N gene) and ssDNA reporter were then added to the tube. The final concentration of Cas12a-gRNA, DNA activator and ssDNA reporter were 20 nM, 1 nM, and 8 μM, respectively. Fluorescence was monitored every 1 min for 30 min while the tube remained at the specified temperature. The insert shows net fluorescence (difference between the fluorescence intensities at 30 min and at time 0).

FIGS. 4A and 4B. (FIG. 4A) Comparison of Cas12a-mediated fluorescence detection (10 min) at room temperature (approximate 23° C.) and 37° C. (FIG. 4B) Cas12a-mediated fluorescence detection after different reaction time of the Cas12a-mediated cleavage of the reporter at the room temperature. The RT-LAMP reaction was designed for targeting the N gene. The positive samples contained 3750 copies of SARS-CoV-2 RNA before the start of RT-LAMP. The negative controls contained all the reagents but no target; the input sample was nuclease-free water instead of the viral RNA.

FIG. 5. Representative images obtained from the detection of the N gene at a range of concentrations (0, 8, 15, 30, 45, 60, 75, 750, 7500, 75000 and 750000 copies/μL). Five μL of sample was used.

FIGS. 6A and 6B. Analysis of the controls (FIG. 6A) and human samples (FIG. 6B) using the prepared assay kit containing dehydrated reagents. The Cas12a reagent mixture containing the RNP complex, MgSO₄ and Tris-HCl was dehydrated on the cap. The RT-LAMP reagent mixture (except primers) was dehydrated at the bottom of the tube. (FIG. 6A) The positive control contained 5 μL of 7500 copies/μL viral RNA as the input sample. The negative control contained 5 μL of water as the sample input. (FIG. 6B) Sample #29 was confirmed negative and sample #72 was confirmed positive using RT-PCR. The positive control (PC) contained 2 μL of 7500 copies/μL of viral RNA. The negative control (NC) contained all the reagents but no target, with the input sample being 2 μL of nuclease-free water.

FIG. 7. Thermal image of a 0.2-mL PCR reaction tube that was heated at the bottom to 62° C. The PCR tube contained 25 μL RT-LAMP reagent mixture at the bottom of the tube and 10 μL of Cas12a reagent mixture inside the cap of the tube. The bottom of the tube was heated and maintained at 62° C. for 30 min, and the image was obtained by using a TG165 Imaging IR Thermometer (FLIR). The image indicates approximately 31° C. near the cap of the tube. The E stands for emissivity.

FIGS. 8A-8D. Testing different concentrations of SARS-CoV-2 RNA using RT-LAMP and SYBR Green I dye detection. (FIG. 8A) RT-LAMP amplification curves from the analyses of the N gene at different concentrations. (FIG. 8B) Time to reach threshold fluorescence from the analysis of the N gene at different concentrations. The threshold fluorescence was set at 100,000 (arbitrary unit). (FIG. 8C) RT-LAMP amplification curves from the analyses of the E gene at different concentrations. (FIG. 8D) Time to reach threshold fluorescence from the analysis of the E gene at different concentrations. The threshold fluorescence was set at 200,000 (arbitrary unit). The RNA samples were extracted from Vero-E6 cell cultures infected with SARS-CoV-2, and the concentrations were previously determined using an RT-qPCR assay.

FIGS. 9A-9F. Amplification of the N gene using RT-LAMP at (FIG. 9A) 62° C., (FIG. 9B) 57° C., (FIG. 9C) 52° C., (FIG. 9D) 47° C., (FIG. 9E) 42° C., and (FIG. 9F) 37° C. The starting sample contained 5 μL of viral RNA at a concentration of 75,000 copies/μL. SYBR Green was used for real-time fluorescence monitoring. The 25 μL of RT-LAMP reaction solution contained 1.4 mM dNTP, 1×Isothermal Amplification Buffer, 8 mM MgSO₄ (including 2 mM MgSO₄ in 1×Isothermal Amplification Buffer), 0.2 μM each of the outer primers (F3 and B3), 1.6 μM each of the inner primers (FIP and BIP), 0.8 μM each of the loop primers (LF and LB), 4 units of RNase inhibitor, 7.5 units of WarmStart® RTx reverse transcriptase, 8 units of Bst 2.0 DNA polymerase, and 5 μL of 75,000 copies/μL viral RNA (as target) or nuclease-free water (as negative control).

FIG. 10. The dNTP inhibits the trans-cleavage activity of Cas12a by interacting with the Mg²⁺. After supplementing additional Mg²⁺ into the reaction, trans-activity of Cas12a was recovered. In Reaction (i), 25 μL of solution A was prepared to simulate the components of RT-LAMP reagent, containing 1×Isothermal Amplification Buffer, 8 mM MgSO₄ (including 2 mM MgSO₄ in 1×Isothermal Amplification Buffer), 0.2 μM each of the outer primers (N gene-F3 and B3), 1.6 μM each of the inner primers (N gene-FIP and BIP), 0.8 μM each of the loop primers (N gene-LF and LB), 4 units of RNase inhibitor, 7.5 units of WarmStart® RTx reverse transcriptase, 8 units of Bst 2.0 DNA polymerase, and 5 nM of N gene dsDNA activator. Solution A was mixed with 10 μL Cas12a reagent, containing 400 nM of RNP complex and 10 μM of ssDNA reporter in 50 mM Tris-HCl buffer (pH=7.9). Reaction (ii) was the same as Reaction (i) except adding additional 1.4 mM of dNTP in the solution A. Reaction (iii) was the same as Reaction (ii) except adding additional 10 mM of MgSO₄ in the Cas12a reagent. These 35 μL mixtures were incubated at 23° C. and their fluorescence was monitored in real-time at 1-min interval.

FIG. 11. Optimization of the Mg²⁺ concentration in the Cas12a regent mixture. 10 μL of Cas12a reagent was prepared and added on the cap, containing 400 nM of RNP complex, 10 μM ssDNA reporter, and various concentrations of Mg²⁺ (10, 20, 40 and 80 mM) in 50 mM of Tris-HCl buffer (pH=7.9). These Cas12a reagents were then mixed with 25 μL of RT-LAMP amplification products. The RT-LAMP reaction was designed for targeting the N gene. The positive samples contained 3750 copies of SARS-CoV-2 RNA before the start of RT-LAMP. The negative controls contained all the reagents but no target; the input sample was nuclease-free water instead of the viral RNA. These mixtures were left at room temperature. These results show that Mg²⁺ at 40 mM is optimum for the activity of Cas12a.

FIG. 12. Optimization of the ssDNA reporter concentration in the Cas12a regent mixture. Ten microliters (10 μL) of Cas12a reagent was prepared and added on the cap, containing 400 nM of RNP complex, 40 mM MgSO₄, and various concentrations of ssDNA reporter (2.5, 5, 10 and 20 μM) in 50 mM of Tris-HCl buffer (pH=7.9). These Cas12a reagents were then mixed with 25 μL of RT-LAMP amplification product. The RT-LAMP reaction was designed for targeting the N gene. The positive samples contained 3750 copies of SARS-CoV-2 RNA before the start of RT-LAMP. The negative controls contained all the reagents but no target; the input sample was nuclease-free water instead of the viral RNA. These mixtures were left at room temperature. The pictures of the reaction tubes were recorded under excitation of UV lamp at 2 min, 4 min, 6 min, 8 min, 10 min and 60 min (Part of result is shown in FIG. 4B). The pictures captured at 10 min are shown in this figure. The fluorescence increases when the ssDNA reporter is increased from 2.5 to 10 μM. Little increase is observed when ssDNA reporter is further increased from 10 to 20 μM. We also noticed marginal increases in background signal of negative controls along with an increase of the concentration of the ssDNA reporter, particularly from 10 to 20 μM. Therefore, we chose to use 10 μM reporter, to achieve the brightest fluorescence and relatively low background.

FIG. 13. Representative images obtained from the detection of the N gene of SARS-CoV-2 at very low copy numbers. The experiment was repeated in four batches. In each batch, samples containing different concentrations of the viral RNA (0, 1, 5, 10, 15 and 30 copies/μL) were analyzed in triplicate. Five microliters (5 μL) of sample was used for each reaction.

FIG. 14. Representative images obtained from the detection of the E gene at a range of concentrations (0, 8, 15, 30, 45, 60, 75, 750, 7500, 75000 and 750000 copies/μL). Five microliters (5 μL) of sample was used for each reaction.

FIGS. 15A-15E. Twelve replicate analyses showing reproducible detection of the N gene of SARS-CoV-2. The positive sample contained 750 copies/μL of RNA extracted from supernatants of infected Vero-E6 cell cultures. The negative sample contained 5 μL of nuclease-free water as the sample input. (FIG. 15A) Pictures taken using a personal smartphone camera. (FIG. 15B and FIG. 15C) Color intensity obtained using the Image J software (NIH). Operation procedures for measuring color intensity were: (1) Open picture in the Image J software; (2) Click the Image-Color-Split Channels; (3) Choose the green channel; (4) Select the entire fluorescent area; (5) Click Analyze-Measure. The measured mean value is referred to as color intensity. The overall color intensities (arbitrary unit) were 232±8 from the 12 positive tests and 127±13 from the 12 negative tests. (FIG. 15D and FIG. 15E) Fluorescence intensity measured using a fluorescence detector (built in with the Thermo Fisher Scientific StepOnePlus™ Real-Time PCR System). Fluorescence intensity was measured after 30 min of RT-LAMP and 10 min of CRISPR Cas12a-mediated reaction. The overall fluorescence intensities (arbitrary unit) were (783±47)×103 from the 12 positive tests and (75±6)×103 from the 12 negative tests.

FIG. 16. Images obtained from the analyses of 5 μL RNA extract from non-infected people (sample #: 1, 19, 39, 51, 61, 71). The N gene of SARS-CoV-2 was detected. The positive control (PC) contained 3750 copies of the N gene at the start of the amplification. The negative control (NC) contained all the reagents but no target, nuclease-free water was used as the input sample instead of the N gene.

### DETAILED DESCRIPTION

The present disclosure relates generally to Isothermal amplification and ambient visualization in a single tube for the detection of SARS-CoV-2 using loop-mediated amplification and CRISPR technology.

In one aspect, there is provided a method of detecting SARS-CoV-2 in a sample from a subject having a SARS-CoV-2 infection, or suspected of having a SARS-CoV-2 infection, or at risk of a SARS-CoV-2 infection, comprising:
subjecting the sample to reverse transcription loop-mediated exponential amplification (RT-LAMP) to amplify a target sequence with said SARS-CoV-2 and generate an RT-LAMP amplicon comprising the target sequence;
contacting the RT-amplicon with a reaction mixture at a reaction temperature for a reaction time, the reaction mixture comprising:
   a guide RNA (gRNA) comprising a polynucleotide sequence complementary to the target sequence;
   a Cas12a protein; and
   a ssDNA reporter molecules comprising a first end having a quencher molecule and a second end having a fluorophore
detecting a signal indicative of amplification of the region of the target nucleic acid molecule,
   wherein, if a signal detected, the subject is determined to have a SARS CoV-2 infection,
   wherein, if a signal is not detected, the subject is determined to not to have a SARS CoV-2 infection.

Severe acute respiratory syndrome (SARS) is a viral respiratory illness caused by a coronavirus called SARS-associated coronavirus (SARS-CoV). SARS-CoV-2 is a new coronavirus that is responsible for the 2020 COVID-19 global pandemic.

The term “RT-LAMP” refers to reverse transcription loop-mediated isothermal amplification (RT-LAMP).

Isothermal amplification is carried out at a constant temperature, and does not require a thermal cycler. The RT-LAMP assay uses a DNA polymerization enzyme with high strand-displacement activity and 4 primers, specifically designed to recognize 6 distinct regions on the target gene, to synthesize large amounts of target viral nucleic acids under a constant temperature, usually between 55-65° C. An additional pair of “loop primers” may be used to further accelerate the reaction. The LAMP reaction yields high amount of amplification products, which can be detected either visually or by simple detectors.

RNAs were mixed with RT-LAMP reagents which included the six required RT-LAMP primers, designated F3, B3, FIP, BIP, LF and LB. Reactions also contained the intercalating fluorescent SYBR Green dye, which yields a fluorescent signal upon DNA binding. DNA synthesis was quantified as the increase in fluorescence over time, which yielded a typical curve describing exponential growth

The term “target sequence” refers to the particular nucleotide sequence of the target nucleic acid that is to be amplified and detected. The target sequence includes the sequences to which oligonucleotide primers hybridize during the nucleic acid amplification process. Where the target nucleic acid is originally single-stranded, the term target sequence will also refer to the sequence complementary to the target sequence as present in the target nucleic acid. Where the target nucleic acid is originally double-stranded the term target-sequence refers to both the sense (+) and antisense (−) strands.

In a specific example, the target sequence is the E gene or N gene of SARS-CoV-2.

Guide RNA (gRNA)” may be used interchangeably with “short guide RNA (sgRNA)” or “single guide RNA (sgRNA). The sgRNA is a short synthetic RNA composed of a “scaffold” sequence necessary for Cas12a-binding and a user-defined ˜20 nucleotide “spacer” or “targeting” sequence which defines the genomic target to be modified. The genomic target of Cas9 can be changed by changing the targeting sequence present in the sgRNA.

The term “Cas protein” refers to a CRISPR-associated protein which is a related protein in the CRISPR system.

The CRISPR-Cas12a system refers to a type II CRISPR/cas gene editing system utilizing a Cas12a (also called Cpf1) nuclease.

The term “PAM” refers to a protospacer-adjacent motif, which is required for Cas12a cleavage.

The term “subject”, as used herein, refers to an animal, and may include, for example, domesticated animals, such as cats, dogs, etc., livestock (e.g., cattle, horses, pigs, sheep, goats, etc.), laboratory animals (e.g., mouse, rabbit, rat, guinea pig, etc.), mammals, non-human mammals, primates, non-human primates, rodents, birds, reptiles, amphibians, fish, and any other animal. The subject may be an infant, a child, an adult, or elderly. In a specific example, the subject is a human.

In some examples, a subject is suspected of having a SARS-CoV-2 infection or is at risk of having or developing a SARS-CoV-2 infection.

A subject suspected of having a SARS-CoV-2 infection refers to subjects who present with clinical or biochemical symptoms associated with a SARS-CoV-2 infection, regardless of whether they have been diagnosed as having the infection.

A subject at risk of having SARS-CoV-2 infection may be a subject that is predisposed to develop an infection. Such a subject can include, for example, a subject with a known or suspected exposure to SARS-CoV-2. A subject at risk of having an infection also can include a subject with a condition associated with impaired ability to mount an immune response to an infectious organism or agent, such as SARS-CoV-2. A subject at risk of having SARS-CoV-2 infection may be a subject was in proximity to a subject having a SARS-CoV-2 infection.

The term “infection” as used herein, refers to a disease or condition attributable to the presence in a host of a foreign organism or agent that reproduces within the host. Infections typically involve breach of a normal mucosal or other tissue barrier by an infectious organism or agent. In a specific example, an infection is a SARS-CoV-2 infection.

The term “sample” or “biological sample”, as used herein, refers to animal or human samples including, without limitation, any biological fluid (blood, bone marrow, plasma, serum, bronchoalveolar washing fluid, urine, nasal secretion, ear secretion, urethral secretion, cerebrospinal fluid, pleural fluid, synovial fluid, peritoneal fluid, sputum, lymph, plasma, ejaculate, lung aspirate, etc.), cell, tissue, organ or portion thereof that contains DNA. A sample can be obtained by conventional methods, using processes known in the state of the art by the person skilled in the art. In a specific example, the sample is a respiratory swab sample. In another example, the sample is a nasopharyngeal swab sample.

In some examples, a sample is used directly (e.g., fresh or frozen), or can be manipulated prior to use, for example, by extraction (for example of nucleic acids), fixation (e.g., using formalin) and/or embedding in wax (such as FFPE tissue samples). Samples also include all samples useful for detection of a pathogen in an environment (such as a clinic or hospital), including but not limited to a water or fluid sample, a food sample, or a surface swab.

In some examples, the gRNA comprises or consists of 41 nucleotides.

In some examples, the gRNA comprises or consists of the nucleotide sequence of SEQ ID NO: 16 or SEQ ID NO: 19.

In some examples, the ssDNA reporter comprises or consists of 8 nucleotides.

In some examples, the ssDNA reporter comprises or consists of the nucleotide sequence/6-FAM/TTATTATT/IABk/.

Examples of the fluorophores include, but are not limited, to fluorescein and derivatives thereof (e.g., fluorescein isothianate (FITC), carboxyfluorescein (FAM), tetrachlorofluorescein (TET), 2′,7′-difluorofluorescein (Oregon Green® 488), Oregon Green@514 carboxylic acid, and a fluorescein with chloro and methoxy substituents (JOE and 6-JOE)); rhodamine derivatives (e.g., tetramethyl rhodamine (TAMRA), tetramethyl rhodamine iso-thiocyanate (TRITC), tetramethylrhodamine (TMR), carboxy-X-rhodamine (ROX), Texas Red (a mixture of isomeric sulfonyl chlorides and sulforhodamine; Invitrogen™) and Texas Red-X (Texas Red succinimidyl ester, which contains an additional seven-atom aminohexanoyl spacer (“X”) between the fluorophore and its reactive group; Invitrogen™) and Rhodamine X); cyanine (Cy) dyes (e.g., Cy3, Cy5 and Cy5.5) and cyanine derivatives (e.g., indocarbocyanine (Quasar® 570, Quasar® 670 and Quasar® 705), Oregon Green@isothiocyanate, and eosin isothiocyanate (EITC)); N-hydroxysuccinimidyl 1-pyrenebutyrate (PYB); N-hydroxysuccinimidyl 1-pyrenesulfonate (PYS); (5-(2′-aminoethyl)aminonaphthalene (EDANS); CAL Fluor® Gold 540, CAL Fluor® Orange 560, Fluor® Red 590, CAL Fluor® Red 610, and CAL Fluor® Red 635 (proprietary fluorophores available from Biosearch Technologies, Inc.); VIC®; HEX® (a 6-isomer phosphoramidite); and NED®.

In some examples, quenchers include, but are not limited to, Black Hole Quencher® dyes (BHQ®-1, BHQ®-2, BHQ®-3); p-(dimethyl aminophenylazo)benzoic acid (DABCYL); Deep Dark Quencher DDQ-1 (Eurogentec); Eosin(2′,4′,5′,7′-tetrabromofluorescein); Eclipse® Dark Quencher (Eurogentec); Iowa Black® Quenchers, e.g., Iowa Black® FQ and Iowa Black® RQ (Integrated DNA Technologies, Inc.); QSY-7, QSY-9 and QSY-21 (Molecular Probes®).

In some examples, fluorophore-quencher pairs include, but are not limited to, fluorescein/DABCYL, EDANS/DABCYL, CAL Fluor® Gold 540/BHQ®-1, Cy3/BHQ-1, FAM/BHQ®-1, TET/BHQ®-1, JOE/BHQ®-1, HEX/BHQ®-1, Oregon Green®/BHQ-1, Cy3/BHQ®-2, Cy5/BHQ-2, ROX/BHQ®-2, TAMRA/BHQ-2, Cy5/BHQO-3, and Cy5.5/BHQ®-3.

In one example, the quencher molecule is Iowa Black® fluorescence quencher and the fluorophore is 6-carboxyfluorescein.

In some examples, RT-LAMP reaction temperature ranges from 55° C. to 70° C. CRISPR-Cas reaction temperature ranges from 20° C. to 50° C. In one example, RT-LAMP reaction temperature is 62° C. and CRISPR-Cas reaction temperature is 23° C.

The terms “polynucleotide”, “nucleic acid” and “oligonucleotide” refers to biopolymers of nucleotides and, unless the context indicates otherwise, includes modified and unmodified nucleotides, and both DNA and RNA. It will be appreciated that nucleic acid whose nucleotide is replaced by an artificial derivative or modified nucleic acid from natural DNA or RNA is also included in the nucleic acid of the present invention insofar as it functions as a template for synthesis of complementary chain.

The term “primer”, “polynucleotide primer” refers to a short polynucleotide that satisfies the requirements that it is able to form complementary base pairing sufficient to anneal to a desired nucleic acid template for use in the methods herein, including amplification reactions such as PCR, LR-PCR, RT-PCR, and RT-LAMP.

A primer may also be modified by attachment of one or more chemical moieties including but not limited to, biotin, a fluorescent tag, a phosphate, or a chemically reactive group.

As used herein the term “amplification” and its variants includes any process for producing multiple copies or complements of at least some portion of a polynucleotide, said polynucleotide typically being referred to as a “template” or, in some cases, as a “target.” The template (or target) polynucleotide can be single stranded or double stranded. Amplification of a given template can result in the generation of a population of polynucleotide amplification products.

The primers are generally isolated.

Method of the invention are conveniently practiced by providing the compounds and/or compositions used in such method in the form of a kit. Such kit preferably contains the composition. Such a kit preferably contains instructions for the use thereof.

To gain a better understanding of the invention described herein, the following examples are set forth. It should be understood that these examples are for illustrative purposes only. Therefore, they should not limit the scope of this invention in anyway.

### EXAMPLE

We have developed a single-tube assay for SARS-CoV-2 in patient samples. This assay combined advantages of isothermal and exponential amplification with clustered regularly interspaced short palindromic repeats (CRISPR) and CRISPR-associated (Cas) enzymes. We report here a strategy of integrating reverse transcription (RT) loop-mediated isothermal amplification (LAMP) with CRISPR-Cas12a to achieve successful analyses of SARS-CoV-2 in a single tube within 40 min. The assay required only a single temperature control (62° C.) and fluorescence was visualized at the room temperature. The RT-LAMP reagents were added to the sample vial, while CRISPR-Cas12a reagents were deposited onto the lid of the vial. After a half-hour RT-LAMP amplification, the tube was inverted and flicked to mix the detection reagents with the amplicon. The sequence-specific recognition of the amplicon by the CRISPR guide RNA and Cas12a enzyme improved specificity. Visible green fluorescence generated by the CRISPR-Cas12a system was recorded using a smartphone camera. Analysis of 100 human respiratory swab samples for the N and/or E gene of SARS-CoV-2 produced 100% clinical specificity and no false positive. Analysis of 50 samples that were detected positive using reverse transcription quantitative polymerase chain reaction (RT-qPCR) resulted in an overall clinical sensitivity of 94%. Importantly this included 20 samples that required 30-39 threshold cycles of RT-qPCR to achieve a positive detection. Integration of the exponential amplification ability of RT-LAMP and the sequence-specific processing by the CRISPR-Cas system into a molecular assay resulted in the improvements in both analytical sensitivity and specificity. The single-tube assay is beneficial for future point-of-care applications.

Global demands and competition for COVID-19 test kits and reagents necessitate the development of alternative assays and diagnostic tools. In addition, the need for thermal cycling for PCR tests makes point-of-care testing and on-site analysis in remote communities challenging. Consequently, there has been much interest in the development of molecular assays using isothermal amplification of nucleic acids.¹²⁻¹⁷ Two isothermal amplification techniques, loop-mediated isothermal amplification (LAMP)¹⁸⁻²¹ and recombinase polymerase amplification (RPA),²²⁻²⁴ have been demonstrated to achieve the detection of SARS-CoV-2 RNA. LAMP is particularly promising for point-of-care applications because it requires only a single enzyme for the exponential amplification.²⁵

Recent advances in clustered regularly interspaced short palindromic repeats (CRISPR) and CRISPR-associated (Cas) proteins have stimulated the application of CRISPR technology and isothermal amplification techniques to the development of diagnostic assays for COVID-19. Broughton et al.²⁶ combined RT-LAMP with CRISPR-Cas12a and developed a CRISPR-based assay for the detection of SARS-CoV-2. Named SARS-CoV-2 DNA endonuclease-targeted CRISPR trans reporter (DETECTR), this assay reduced the analysis time to <40 min, eliminated thermal cycling, and produced a color readout visible on lateral flow strips. The assay is potentially useful for point-of-care applications, although it required three steps: RT-LAMP amplification, CRISPR-mediated cleavage of reporters, and lateral flow display of results. The assay is conducted in two separate tubes under three temperature conditions: 62° C., 37° C., and room temperature. The use of two controlled temperatures (62° C. and 37° C.) increases the device requirement for the assay. Opening tubes to transfer reaction reagents and products increases the risk of potential contamination, which compromises the applicability of the assay for point-of-care testing.

With the ultimate goal of simplifying the assay for point-of-care testing, we addressed the issues impeding RT-LAMP and CRISPR reactions in a single tube. We examined the apparent incompatibility between RT-LAMP and CRISPR, namely differences in reaction temperatures and changes in the pH and Mg²⁺ concentration in the process of LAMP. We report here an improved assay that incorporates RT-LAMP and CRISPR technology and demonstrate its application for the detection of SARS-CoV-2 in a single tube. In principle, the target sequence of viral RNA is reverse transcribed and exponentially amplified by RT-LAMP and the specific amplicons are recognized by the CRISPR guide RNA and Cas12a enzyme. The integration of RT-LAMP with CRISPR takes advantage of both target amplification and sequence-specific recognition, resulting in improvements in both analytical sensitivity and specificity. Operationally, all necessary reactions are performed in a single closed tube and the assay only needs a single temperature control (62° C.). The improvement achieved by reducing the number of steps and simplifying the assay requirement and thus eliminating the risk of contamination during the assay represents an important step toward point-of-care applications.

Joung et al.⁴ reported “detection of SARS-CoV-2 with SHERLOCK one-pot testing”, built on the success of SHERLOCK (specific high-sensitivity enzymatic reporter unlocking) technology. The assay combined RT-LAMP with Cas12b and detected the N gene as the target. Recognizing that “LAMP operates at 55 to 70° C. and requires a thermostable Cas enzyme”, the authors generated and used Cas12b enzyme from Alicyclobacillus acidiphilus (AapCas12b) and identified the best combination of LAMP primers and guide RNAs to target the N gene. They tested “202 SARS-CoV-2-positive and 200 SARS-CoV-2-negative nasopharyngeal swab samples obtained from patients”, and conclude that the assay “is suited for use in low-complexity clinical laboratories”.

The assay we report here targets both the E gene and N gene, while Joung et al.⁴ detected the N gene. Our assay uses commercially available Cas12a enzyme and this enzyme system requires only a short (41 nt) guide RNA (gRNA), whereas Cas12b used by Joung et al.⁴ is not commercially available and it requires a longer (111 nt) single guide RNA (sgRNA) composed of both crRNA and tracrRNA. The longer sgRNA could have a risk of “partial overlap between the sgRNA and one of the primers for LAMP”, which could “contribute to sporadic Cas12b collateral activity” and “occasional false positive results”.⁴ The 41-nt gRNA we use for the Cas12a system does not have sequence overlap with any of the LAMP primers.

### EXPERIMENTAL SECTION

Viral RNA of SARS-CoV-2

The original SARS-CoV-2 virus strain (SARS-CoV-2/CANADA/VIDO 01/2020) was obtained from the University of Saskatchewan, Canada (a kind gift from Dr. Darryl Falzarano). SARS-CoV-2 was produced from the infection of Vero-E6 cells and the amount of viral RNA was quantified using RT-qPCR for detecting the N gene (with N2 primers designed by the U.S. CDC).

Clinical Specimens and Extraction of RNA

One hundred (100) respiratory swab specimens were collected by the Alberta Precision Laboratories (Canada). RNA was extracted using one of three platforms: the easyMAG® (BioMerieux, Quebec, Canada), the KingFisher Flex automated extraction and purification systems (Thermo Fisher Scientific) or the Hamilton STARlet automated extractor (Hamilton, Reno, Nev., USA). The specimen input was 200 μL and the purified nucleic acid was extracted into 100 μL. An aliquot of 1-10 μL of the RNA extract was used as the sample input for the detection of the N gene and E gene of SARS-CoV-2.

Reverse Transcription Loop-Mediated Exponential Amplification (RT-LAMP)

Sequences of the amplified regions of the N gene and E gene of SARS-CoV-2, along with sequences of the primer sets for RT-LAMP, are summarized in Table 1. All primers were synthesized by Integrated DNA Technologies (IDT, San Diego, Calif.).

Briefly, a 25-μL RT-LAMP reaction solution contained 1.4 mM deoxynucleotide (dNTP, NEB), 1×Isothermal Amplification Buffer (NEB), 0.2 μM each of the outer primers (F3 and B3), 1.6 μM each of the inner primers (FIP and BIP), 0.8 μM each of the loop primers (LF and LB), 4 units of RNase inhibitor (Invitrogen), 7.5 units of WarmStart® RTx reverse transcriptase (NEB), 8 units of Bst 2.0 DNA polymerase (NEB), and 1-10 μL of sample or nuclease-free water (as negative control). RT-LAMP reactions were performed at 62° C. for 30 min.

CRISPR Cas12a-Mediated Fluorescence Detection

The sequences of the guide RNAs (gRNAs), recognizing the specific sequences of the RT-LAMP amplicons, are listed in Table 2. Cas12a-mediated trans-cleavage of the ssDNA reporter, for the purpose of fluorescence detection, was carried out at room temperature (approximately 23° C.) for 10 min. The EnGen© Lba Cas12a enzyme (NEB) at 1 μM concentration was pre-incubated with 1.25 μM of gRNA in 1×NEBuffer 2.1 (NEB) to form the ribonucleoprotein (RNP) complex. An aliquot of the RNP complex solution was placed inside the cap of a PCR tube. The optimum concentrations of the Cas12a reaction reagents were 400 nM of the RNP complex, 40 mM MgSO₄, and 10 μM ssDNA reporter in 50 mM Tris-HCl buffer (pH=7.9).

RT-LAMP and CRISPR Cas12a-Mediated Ambient Visualization in a Single Tube for the Detection of SARS-CoV-2

A 25-μL mixture of the RT-LAMP reaction reagents was added at the bottom of a 0.2-mL PCR tube and 10 μL of the Cas12a reaction solution was added inside the cap of the tube. An aliquot (2-10 μL) of RNA extract sample was added into the bottom of the tube, mixing with the RT-LAMP reagents. The tube was then gently capped, preventing the Cas12a reagent from falling into the tube. The tube was placed in a temperature controller (heating block) and the bottom of the tube was maintained at 62° C. for 30 min to perform RT-LAMP. While the bottom of the tube was at 62° C., the temperature in the cap was measured to be 31° C. After 30 min of RT-LAMP, the tube was removed from the temperature controller, and the subsequent Cas12a-mediated detection step was performed at the room temperature. Inverting and wrist-flicking the tube mixed the Cas12a reagents with the RT-LAMP amplicons. The tube was left at the room temperature for 10 min. Green fluorescence was visualized under the excitation of a handheld UV lamp and a photo was taken using a personal smartphone. The total time for the analysis of an RNA extract was 40 min, including 30 min for RT-LAMP and 10 min for the Cas12a-mediated detection.

Assay Kit for RT-LAMP and CRISPR Cas12a-Mediated Ambient Visualization in a Single Tube

An assay kit was prepared to consist of a 0.2-mL PCR tube and a vial of rehydration buffer solution containing primers and the reporter. The PCR tube was prepared to contain dried (lyophilized) reagents for the assay. The mixture of the RT-LAMP reaction reagents, except the primers, was added at the bottom of the PCR tube. This mixture was composed of 35 nmol of dNTP, 1×Isothermal Amplification Buffer, 150 nmol of MgSO₄, 4 units of RNase inhibitor, 7.5 units of WarmStart® RTx reverse transcriptase, 8 units of Bst 2.0 DNA polymerase and approximate 5 μmol of D-(+)-trehalose dihydrate (Sigma). The RNP complex (22 μmol) for the Cas12a-mediated reaction, 400 nmol of MgSO₄, and 500 nmol of Tris-HCl were added inside the cap of the tube. The tube was placed in a vacuum desiccator for 2 h to dry the reagents. After the reagents were dry, the tube was capped and stored at 4° C. until use. The vial of rehydration buffer contained the primers for RT-LAMP and 5 μM of ssDNA reporter for the Cas12a-mediated detection. The primers in the buffer solution can be customized according to the target of analysis.

Analysis of Clinical Specimens

Clinical samples were analyzed randomly in batches, using the method of RT-LAMP amplification and CRISPR Cas12a-mediated detection (Table 3). Each batch of analysis included a positive control and a negative control. The positive control contained 2 μL or 10 μL of 750 copies/μL of viral RNA extracted from supernatants of Vero-E6 cell cultures. The negative control contained all the reagents but no target, with the input sample being 2 μL or 10 μL of nuclease-free water. Samples were also analyzed using RT-PCR (Table 4 and Table 5).

Results and Discussion

Principle and Operation of RT-LAMP Amplification and CRISPR-Mediated Visualization in a Single Tube for the Detection of SARS-CoV-2

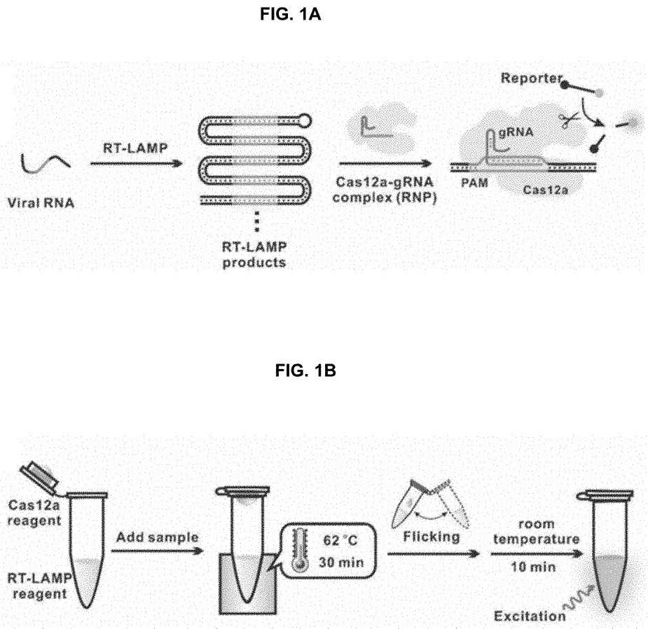

The principle and operation of the assay that incorporates RT-LAMP amplification with CRISPR Cas12a-mediated detection are shown in FIG. 1. Briefly, specific sequences of the E gene and N gene from SARS-CoV-2 (Table 1) are reverse transcribed and amplified using RT-LAMP. The target sequence of the resulting amplicon is recognized by a pre-designed guide RNA (gRNA) and interacts with the Cas12a-gRNA ribonucleoprotein (RNP) complex. Binding of the RNP to the specific amplicon activates the trans-cleavage activity of the Cas12a enzyme, resulting in cleavage of a short (8 nt) ssDNA reporter that is labeled at either end with a fluorophore/quencher pair. Cleavage of the reporter releases the quencher, allowing the fluorophore to fluoresce. The combination of RT-LAMP with CRISPR Cas12a takes advantage of the isothermal exponential amplification ability of RT-LAMP and the sequence recognition property and multiple turn-over enzyme activity of the CRISPR Cas12a system, resulting in improvements in both sensitivity and specificity of the assay.

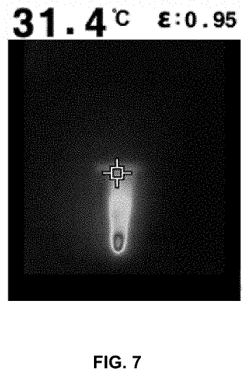

In practice, performing both RT-LAMP and CRISPR Cas12a reactions in a single tube is technically challenging because CRISPR Cas12a has been reported to perform at around 37° C.,⁵ whereas the optimum reaction temperature for RT-LAMP is 60-65° C. To overcome the temperature incompatibility between the Cas12a-mediated reaction and RT-LAMP, we studied the effects of temperature and other conditions on RT-LAMP and CRISPR Cas12a-mediated reactions and developed a new approach of integrating RT-LAMP with CRISPR Cas12a in a single tube (FIG. 11B). Briefly, a 25-μL mixture of reagents for RT-LAMP is placed at the bottom of a 0.2-mL PCR tube, and a 10-μL mixture of reagents for the CRISPR Cas12a-mediated detection is placed inside the cap of the PCR tube. A sample is added to the bottom of the tube and the cap is carefully closed. The tube is placed in a heater where the bottom of the tube is maintained at 62° C., the optimum temperature for the RT-LAMP reaction, whereas the temperature on the cap only reaches to about 31° C. (FIG. 7). After 30 min of RT-LAMP, simple inverting and wrist-flicking the tube mixes the Cas12a reagents with the amplicon generated by RT-LAMP. The CRISPR Cas12a-mediated recognition of the target and cleavage of the reporter result in the generation of bright green fluorescence, visible with excitation of a handheld ultraviolet (UV) lamp. The same principle and operation apply when the assay is performed using the dehydrated reagent kit. The only difference is that the RT-LAMP primers and fluorescent reporter are in the buffer solution, and the remaining RT-LAMP reagents are dried in the bottom of the tube and the Cas12a-gRNA RNP is dried inside the cap.

This assay has three main advantages over other isothermal amplification assays. First, the CRISPR Cas12a-mediated detection improves the detection specificity. Second, the assay requires only a single controlled temperature (62° C.). Because isothermal amplification has a minimal requirement for temperature control, the assay is more amenable for future point-of-care applications. Third, the entire assay is performed in a single tube. After the addition of sample, there is no need to open the tube, avoiding any cross-contamination of other samples by the amplicon.

Primers for RT-LAMP and gRNAs for Cas12a

We chose to detect both the E and N genes of SARS-CoV-2 because the E gene is highly conserved among all beta coronaviruses, and the N gene can be used to differentiate SARS-CoV-2 from other coronaviruses. Our primer sets for RT-LAMP targeted the same gene sequences as those of the previously established RT-PCR assays for the E region (Charite Virology, Germany)⁶ and for the N2 region (US CDC).⁷ We used the gRNA for the E gene assay to possess a broad specificity for SARS-like coronaviruses, such as SARS-CoV-2, SARS-CoV, and bat SARS-like coronavirus (bat-SL-CoVZC45). The gRNA for the N gene assay was designed to specifically recognize the N gene of SARS-CoV-2. Sequences of the primers and gRNAs are summarized in Tables 1 and 2.

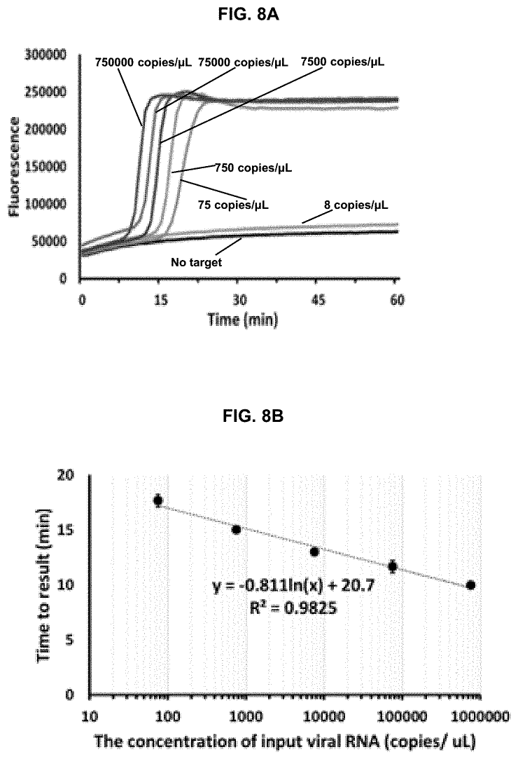

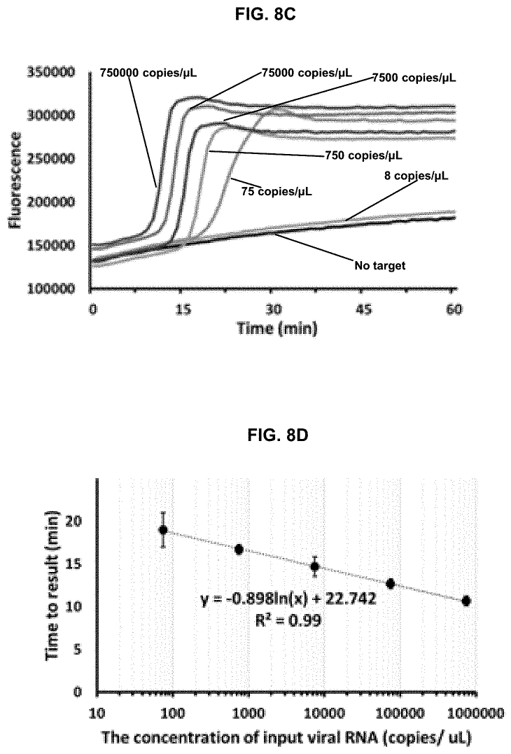

Using RT-LAMP, we tested a series of solutions containing a range of concentrations of viral RNA, from 8 to 750,000 copies per μL. When amplification of RT-LAMP for the N and E genes was monitored in real-time using SYBR Green detection, a plateau was reached before 30 min (FIG. 8). To ensure sufficient amplification of RNA, we allowed 30 min for RT-LAMP.

Improving Specificity Using CRISPR Cas12a-Mediated Detection

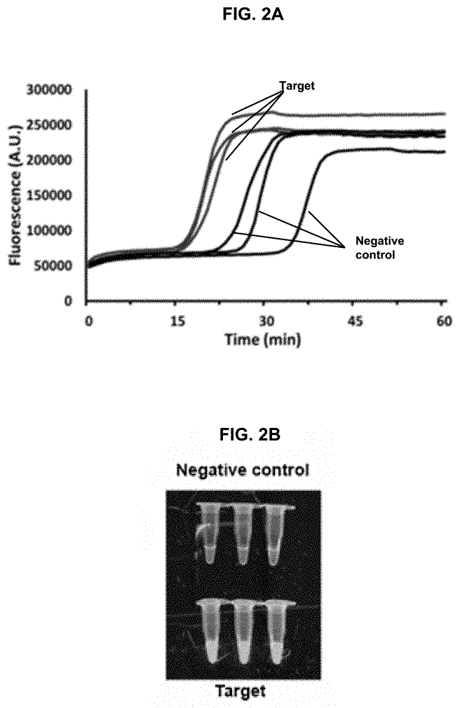

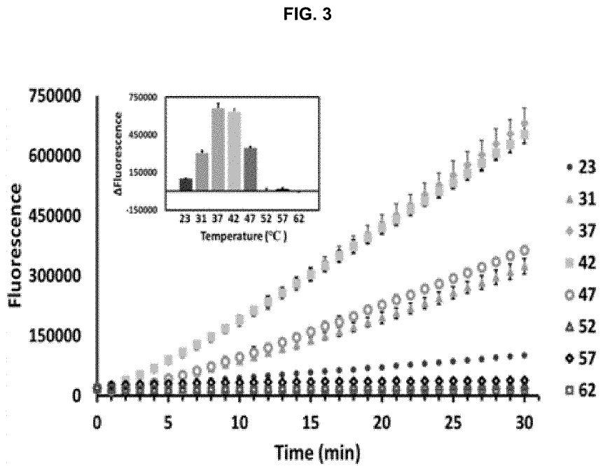

To examine whether the CRISPR-mediated detection improved the specificity, we compared the real-time fluorescence detection using the SYBR Green dye with the CRISPR Cas12a-mediated detection of the RT-LAMP products (FIG. 2). When SYBR Green was used for detection, two of the triplicate negative controls also produced fluorescence, at later times of the amplification. These false-positive results from the negative controls are due to non-specific amplifications. However, when the CRISPR-Cas12a system was used for the detection of the same RT-LAMP reaction products, there was no fluorescence from any of the negative controls (FIG. 2). These results show that the specificity is improved by incorporating the CRISPR-mediated detection with the RT-LAMP assay. Although the conventional SYBR Green detection can provide real-time monitoring of nucleic acids produced by RT-LAMP, non-specific amplification products can potentially lead to false positive results especially in a limited resource setting. Incorporation of the CRISPR-mediated detection overcomes this problem by sequence-specific recognition of the amplicon. Binding of the pre-designed gRNA to the complementary sequence of the target amplicon activates the Cas12a system to generate fluorescence signals. Non-specific amplification products are not recognized by the gRNA and thus the reporter is not cleaved to fluoresce.

Issues of Different Temperatures Required for the RT-LAMP and Cas12a Reactions

Integrating both RT-LAMP and Cas12a reactions in a single tube would take advantages of both isothermal amplification by RT-LAMP and improved specificity of the Cas12a-mediated detection. We initially mixed all the reagents, including primers, dNTP, reverse transcriptase, and polymerase for RT-LAMP with Cas12a, gRNA, and ssDNA reporter, and performed reactions at different temperatures. We maintained the tubes at 62, 57, 52, 47, 42, 37, and 23° C., and monitored fluorescence. However, none of the experiments were successful. We reasoned that the main problem was the incompatible temperatures required for RT-LAMP and Cas12a reactions.

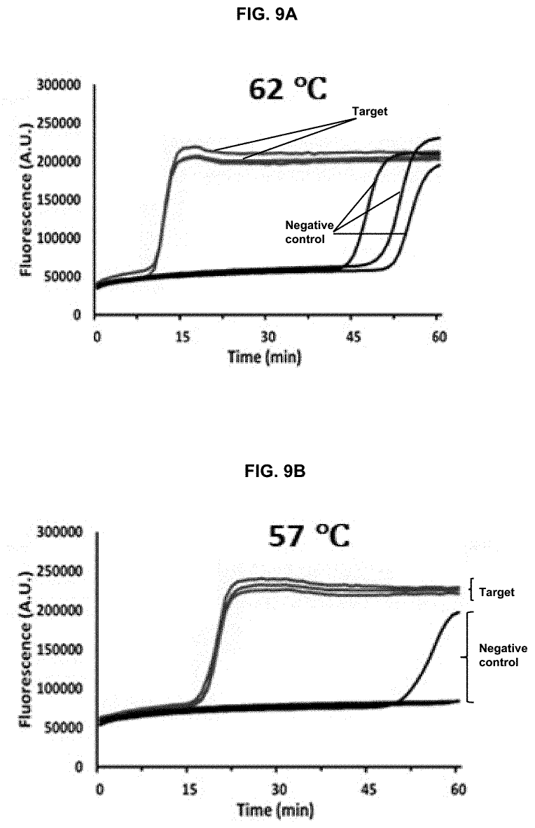

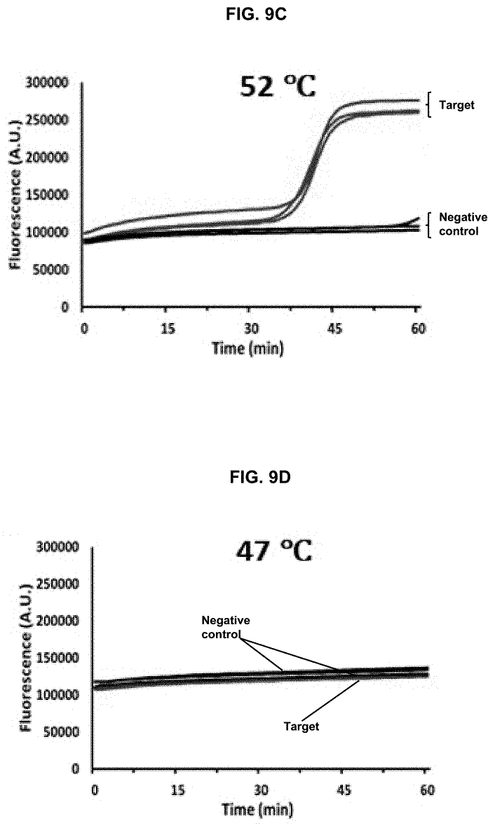

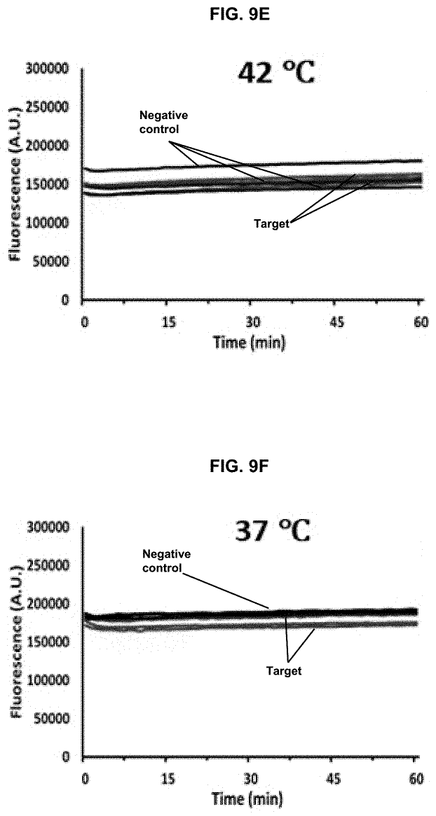

We tested the amplification of the N gene using RT-LAMP at different temperatures (62, 57, 52, 47, 42 and 37° C.) and monitored generation of amplification products in real-time using SYBR Green dye (FIG. 9). As expected, RT-LAMP carried out at 62° C. resulted in the most efficient amplification, needing less than 15 min to reach the exponential amplification phase. Although reactions at lower temperatures (57° C. and 52° C.) also generated products, the time needed to reach exponential amplification was prolonged: 20 min at 57° C. and more than 40 min at 52° C. When the RT-LAMP was conducted at 47, 42, or 37° C., no amplification was observed even after 60 min.

We also examined the ability of Cas12a to trans-cleave ssDNA report at various temperatures: 62, 57, 52, 47, 42, 37, 31, and 23° C. (FIG. 3). These results indicate variable levels of the enzyme activity at 23-47° C., peaking at 37° C., and negligible activity above 52° C. Therefore, the barrier to integrating RT-LAMP amplification with Cas12a-mediated detection was that the reaction temperature for RT-LAMP amplification (52-62° C., FIG. S4) was incompatible with Cas12a-mediated detection (23-47° C., FIG. 3).

Integrating RT-LAMP and Cas12a-Mediated Detection in a Single Tube

To overcome the problem of temperature incompatibility, we designed a two-step operation to combine the entire assay in a single tube (FIG. 1B). We placed a mixture of the Cas12a enzyme, gRNA, and ssDNA reporter inside the cap of a common PCR tube. The temperature of the cap was approximately 31° C. (FIG. 7) during the RT-LAMP reaction that took place at 62° C. in the bottom of the tube. Thus, the stability of the Cas12a reagents inside the cap was maintained.

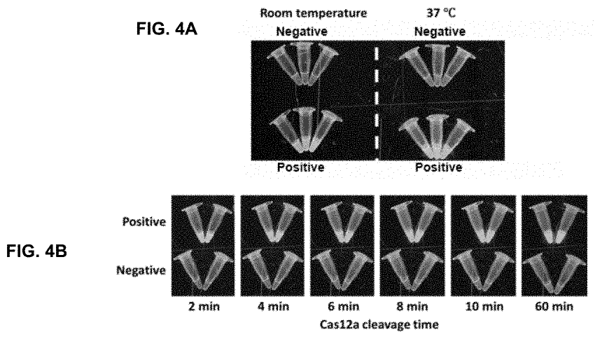

We compared the Cas12a-mediated fluorescence detection at room temperature (approximate 23° C.) and 37° C. (FIG. 4A). After 10 min, similar fluorescence signals were visible at both temperatures. Therefore, there is no need for a controlled temperature of 37° C. Detection at room temperature simplifies the assay for potential on-site applications.

We determined the reaction time needed for the Cas12a-mediated fluorescence detection at room temperature (FIG. 4B). Fluorescence is clearly visible from the positive reactions as rapidly as 2 min after mixing the RT-LAMP product with the Cas12a reagents. There is no significant increase in fluorescence intensity beyond 10 min of Cas12a-mediated reactions. We subsequently chose 10 min of the Cas12a-mediated reaction time for the assay.

Optimization for RT-LAMP and Cas12a Reactions

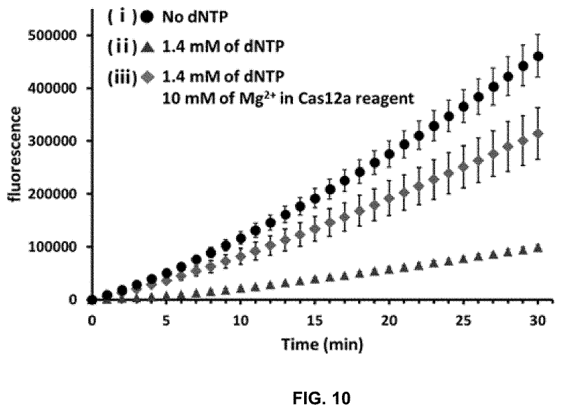

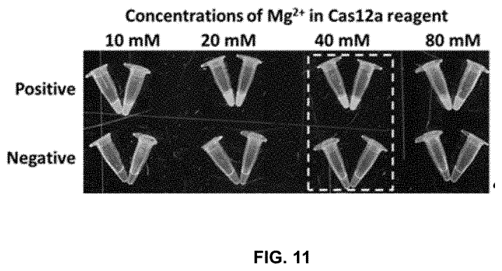

Since the reagents are initially placed in two compartments of a single tube, an added benefit is the ability to fine-tune the RT-LAMP and Cas12a-mediated reaction conditions independently for compatibility and optimum performance. This optimization is particularly important because the available concentration of Mg²⁺ and the pH change as the RT-LAMP reaction progresses. We found that RT-LAMP decreased the concentration of Mg²⁺ to a level that was insufficient for the activity of Cas12a. Either the interaction of dNTP with Mg²⁺ decreased the concentration of Mg²⁺ available for the subsequent Cas12a-mediated reaction,⁸ or pyrophosphate generated during RT-LAMP could form magnesium phosphate precipitates,⁹ decreasing the concentration of Mg²⁺ available for the subsequent Cas12a-mediated reaction. Addition of Mg²⁺ to the Cas12a reaction mixture could regain the activity of Cas12a (FIG. 10). We therefore added Mg²⁺ into the cap containing the Cas12a reagent mixture to compensate for the loss of Mg²⁺ during the RT-LAMP reaction. We found that 40 mM Mg²⁺ in the Cas12a reagent mixture was sufficient to compensate for the loss of Mg²⁺ and ensure the activity of Cas12a (FIG. 11).

Protons liberated during RT-LAMP reactions decreased the pH of the reaction mixture from 8.8 at the beginning of the reaction to 6.0 at the end of RT-LAMP.¹⁰ To ensure the activity of Cas12a in the subsequent reactions, we compensated for changes in pH. Placing the Cas12a-mediated reaction reagents separate from the RT-LAMP reagents facilitated pre-adjustment of the pH in the Cas12a reagent mixture to compensate for changes that occurred during the RT-LAMP process. We determined that 50 mM Tris-HCl buffer (pH 7.9) in the Cas12a reagent mixture was sufficient to compensate for the pH changes during RT-LAMP and to achieve the optimum pH condition for the trans-cleavage activity of the Cas12a system.

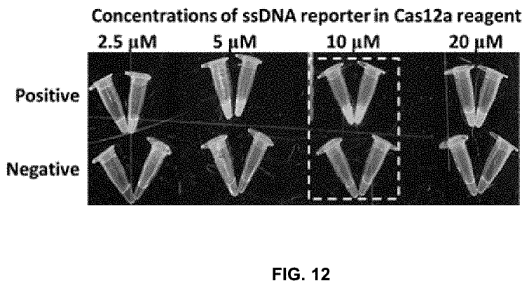

Any unused primers after RT-LAMP can compete with the ssDNA reporter and serve as substrates for the trans-cleavage activity of Cas12a. Therefore, we optimized the concentration of ssDNA reporter in the Cas12a reagent to reduce the effect of unused primers and to ensure the generation of sufficient fluorescence for visualization. We found that 10 μM reporter was optimum for achieving the brightest fluorescence with low background (FIG. 12).

Limit of Detection and Reproducibility

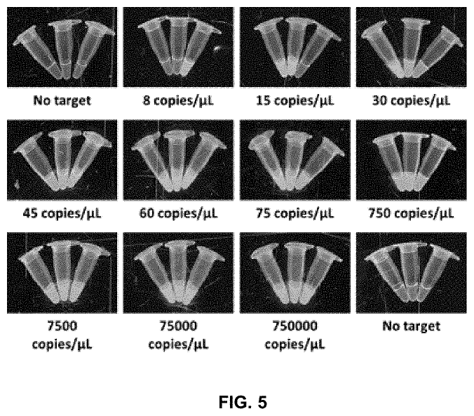

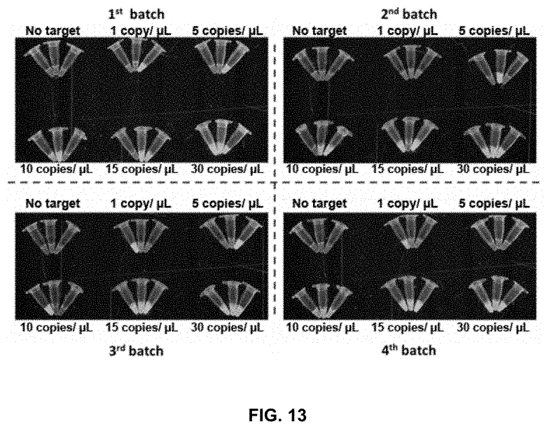

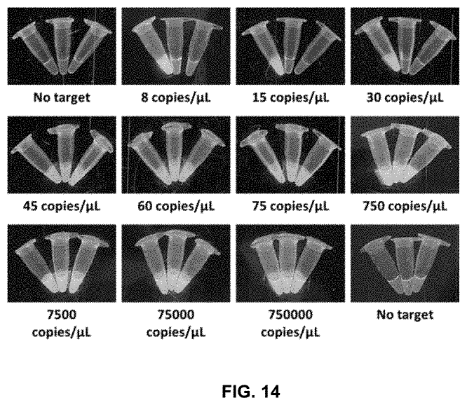

We detected the N gene and E gene of SARS-CoV-2 at a wide range of RNA concentrations, from 1 copy/μL to 750000 copies/μL. SARS-CoV-2 RNA was extracted from supernatants of Vero-E6 cell cultures, and the concentrations of SARS-CoV-2 RNA were measured using RT-qPCR targeting the N gene. Samples containing viral RNA concentrations 30 copies/μL or higher consistently gave positive results from the detection of the N gene (FIG. 5). Samples that contained 1 copy/μL (5 copies for 5 μL sample input) of the target gave occasional positive results (3 out of 12) (FIG. 13); however consistently accurate detection was achieved when the concentration was above 30 copies/μL (150 copies total). True positive rates from 12 replicate analyses were 100% (30 copies/μL), 83% (15 copies/μL), 67% (10 copies/μL), 25% (5 copies/μL), and 25% (1 copy/μL) (Table 6). Random sampling errors from analysis of small numbers of molecules probably contribute to the observed detection rates. Similar random sampling errors also occur in other assays, e.g., RT-qPCR (Table 7). We report a detection limit of 30 copies/μL (150 copies), for a consistent 100% true positive rate. Similar results were obtained from the detection of the E gene, where consistently positive results were obtained when the concentration was higher than 45 copies/μL (FIG. 14).

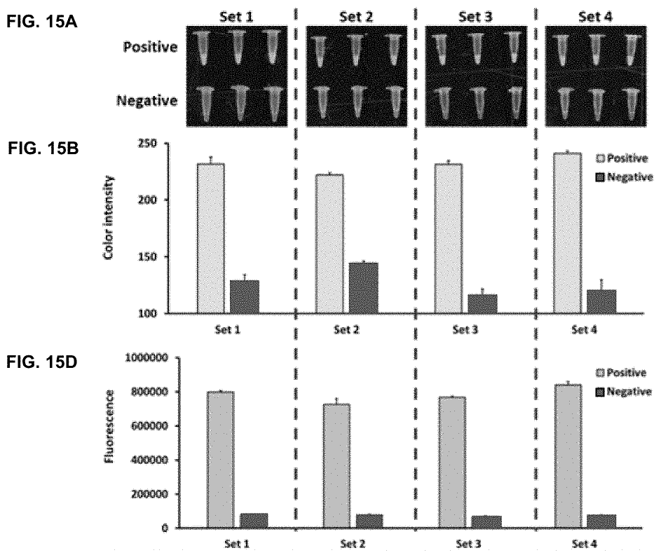

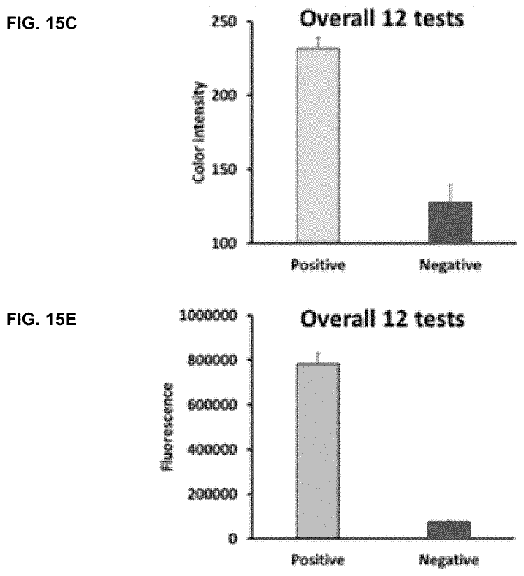

We determined the reproducibility by conducting 12 replicate analyses of the N gene in a sample that contained 750 copies/μL of SARS-CoV-2 RNA and a negative control (nuclease-free water) (FIG. 15). Relative standard deviation (RSD) was 6-7% on the basis of fluorescence intensity measurements and 3-10% from measurements of digitized color intensity.

Analysis of Clinical Specimens

We carried out our assay on 100 clinical samples, consisting of 50 clinical negative and 50 SARS-CoV-2 positive samples. Results from the analysis of multiple batches of clinical samples on multiple days are summarized in Table 3. Overall, we accurately assessed 50 true negatives, 47 true positives, and 3 false negatives from a total of 100 clinical samples. The three false negative samples contained very low concentrations of the target RNA, requiring 33-39 threshold cycles of RT-qPCR to achieve a positive detection. These results demonstrate an overall clinical specificity of 100% (detecting all 50 negatives) and an overall clinical sensitivity of 94% (detecting 47 positives of 50 total positive samples).

The 50 positive samples contained a wide range of viral RNA concentrations, according to the RT-qPCR analyses of the E gene (Ct ranging from 11.7 to 39.2). Of 30 samples that had Ct values below 30, all were positively detected using both the N and E genes, equivalent to a clinical sensitivity of 100% (detecting all 30 positives). Our assay was also successful in correctly detecting 17/20 positives that required more than 30 cycles to be positively detected by RT-qPCR.

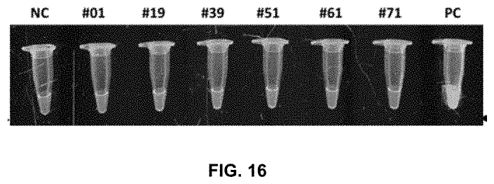

Analyses of all 50 negative samples for both the N gene and E gene consistently yielded negative results. To ensure that our detection of negative signal was not due to insufficient samples, we increased the sample amount from 2 μL to 5 μL and repeated the analysis. Replicate analyses of 6 representative samples still resulted in no signal from these true negative samples (FIG. 16). There was no false positive from the analysis of any of the 100 samples, confirming the 100% clinical specificity of the assay.

Analysis Using a Single-Tube Assay Kit

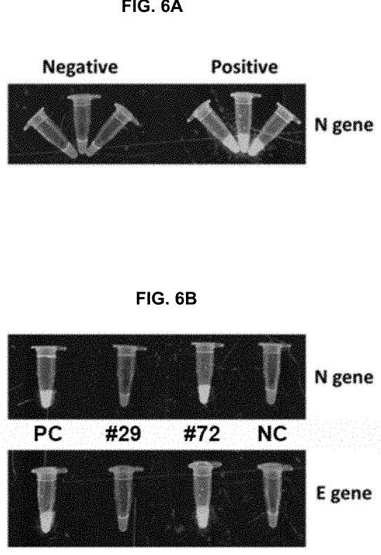

To make the assay kit convenient for shipping and to demonstrate its potential for on-site applications, we have prepared PCR tubes containing dried reagent mixtures and conducted the assay after rehydrating the reagents. A main benefit is that the prepared tubes can be easily shipped to the field and used conveniently to conduct the assay. Our results from the analysis of controls (FIG. 6A) and representative samples (FIG. 6B) confirmed successful detection of SARS-CoV-2 using the prepared tube and reagent kit.

### CONCLUSION

Our assay for the detection of SARS-CoV-2 has two appealing features: 1) conducting CRISPR-Cas detection at room temperature thus only requiring a single controlled temperature for isothermal amplification, and 2) integration of isothermal amplification and subsequent CRISPR-Cas detection in a single tube, which simplifies the operation and eliminates the risk of contamination during the assay. These features are a significant advance in the application of isothermal amplification techniques to point-of-care applications and on-site analysis. The RT-LAMP-Cas12a assay is not limited to the detection of SARS-CoV-2. We envision application of the assay for other infectious agents through simply altering primers to target other nucleic acids.

### REFERENCES

(1) Feng, W.; Newbigging, A. M.; Le, C.; Pang, B.; Peng, H.; Cao, Y.; Wu, J.; Abbas, G.; Song, J.; Wang, D. B.; Cui, M.; Tao, J.; Tyrrell, D. L.; Zhang, X. E.; Zhang, H.; Le, X. C. Molecular Diagnosis of COVID-19: Challenges and Research Needs. Anal. Chem. 2020, 92 (15), 10196-10209.
(2) Zhao, Y.; Chen, F.; Li, Q.; Wang, L.; Fan, C. Isothermal Amplification of Nucleic Acids. Chem. Rev. 2015, 115 (22), 12491-545.
(3) Broughton, J. P.; Deng, X.; Yu, G.; Fasching, C. L.; Servellita, V.; Singh, J.; Miao, X.; Streithorst, J. A.; Granados, A.; Sotomayor-Gonzalez, A.; Zorn, K.; Gopez, A.; Hsu, E.; Gu, W.; Miller, S.; Pan, C. Y.; Guevara, H.; Wadford, D. A.; Chen, J. S.; Chiu, C. Y. CRISPR-Cas12-based detection of SARS-CoV-2. Nat. Biotechnol. 2020, 38, 870-874.
(4) Joung, J.; Ladha, A.; Saito, M.; Kim, N. G.; Woolley, A. E.; Segel, M.; Barretto, R. P. J.; Ranu, A.; Macrae, R. K.; Faure, G.; Ioannidi, E. I.; Krajeski, R. N.; Bruneau, R.; Huang, M. W.; Yu, X. G.; Li, J. Z.; Walker, B. D.; Hung, D. T.; Greninger, A. L.; Jerome, K. R.; Gootenberg, J. S.; Abudayyeh, O. O.; Zhang, F. Detection of SARS-CoV-2 with SHERLOCK One-Pot Testing. N. Engl. J. Med. 2020, DOI: 10.1056/NEJMc2026172.
(5) Chen, J. S.; Ma, E.; Harrington, L. B.; Da Costa, M.; Tian, X.; Palefsky, J. M.; Doudna, J. A. CRISPR-Cas12a target binding unleashes indiscriminate single-stranded DNase activity. Science 2018, 360 (6387), 436-439.
(6) World Health Organization. Diagnostic Detection of Wuhan Coronavirus 2019 by Real-time RT-PCR. 2020, https://www.who.int/docs/default-source/coronaviruse/protocol-v2-1.pdf (accessed 2020-01-17).
(7) Center for Disease Control and Prevention. Research Use Only 2019-Novel Coronavirus (2019-nCoV) Real-time RT-PCR Primers and Probes. 2020, https://www.cdc.gov/coronavirus/2019-ncov/lab/rt-pcr-panel-primer-probes.html (accessed 2020-06-06)
(8) Son, T. D.; Roux, M.; Ellenberger, M. Interaction of Mg²⁺ ions with nucleoside triphosphates by phosphorus magnetic resonance spectroscopy. Nucleic Acids Res. 1975, 2 (7), 1101-10.
(9) Mori, Y.; Nagamine, K.; Tomita, N.; Notomi, T. Detection of loop-mediated isothermal amplification reaction by turbidity derived from magnesium pyrophosphate formation. Biochem. Biophys. Res. Commun. 2001, 289 (1), 150-4.
(10) Tanner, N. A.; Zhang, Y.; Evans, T. C., Jr. Visual detection of isothermal nucleic acid amplification using pH-sensitive dyes. Biotechniques 2015, 58 (2), 59-68.

Associated Content

Supporting Information

Additional Information on the Preparation of SARS-CoV-2 and its Viral RNA

The original SARS-CoV-2 virus strain (SARS-CoV-2/CANADA/VIDO 01/2020) was obtained from the University of Saskatchewan, Canada. SARS-CoV-2 was produced from the infection of Vero-E6 cells at a multiplicity of infection (MOI) of 0.01 for 48 h, followed by harvesting the supernatant. The amount of virus in the supernatant was assessed by a plaque assay and by RT-quantitative PCR (RT-qPCR). Plaque assays were performed as follows: Vero-E6 cells were infected with serially diluted supernatant in infection medium consisting of Dulbecco's Modified Eagle's Medium (DMEM) supplemented with 1×non-essential amino acids (Gibco), 10 mM 4-(2-hydroxyethyl)-1-piperazineethanesulfonic acid (HEPES), 2% fetal bovine serum, 50 IU/mL penicillin, 50 IU/mL streptomycin. After 1 h, the infecting medium was removed and monolayers were overlaid with Minimum Essential Medium (MEM) supplemented with 10 mM HEPES and 1.2% avicel RC-591 (DuPont). After 48 h, cells were fixed in 10% formaldehyde and stained by 0.5% (w/v) crystal violet. Plaques were counted. The amount of viral RNA was quantified as follows: Cell supernatants (140 μL) were collected at various time points after infection, and RNA was isolated by using the QIAmp Viral RNA Mini kit (Qiagen). Reverse transcription was carried out by using Superscript IV Vilo master mix (Invitrogen). RT-qPCR for detecting the N gene of RNA was performed using Taqman fast Master mix containing 2 μL of cDNA. The primers and Taqman probe for the N gene (N2 primers) were designed by the United States Center for Disease Control and Prevention (CDC). A standard curve was generated using dilutions of positive control standards from CDC (IDT Cat #10006625).

Additional Information on Reverse Transcription Loop-Mediated Exponential Amplification (RT-LAMP)

Sequences of the amplified regions of the N gene and E gene of SARS-CoV-2, along with sequences of the primer sets for the RT-LAMP reactions, are summarized in Supporting Information Table 1. The matching colors and underlines denote complementary sequences. The primer set designed for amplifying each gene (N or E) recognizes eight regions in the target sequence. All primers were synthesized by Integrated DNA Technologies (IDT, San Diego, Calif.).

The procedures of RT-LAMP were based on the protocol of New England BioLabs (NEB, www.neb.com/protocols/2014/10/09/typical-rt-lamp-protocol) with minor modifications. Briefly, a 25 μL RT-LAMP reaction solution contained 1.4 mM deoxynucleotide (dNTP, NEB), 1×Isothermal Amplification Buffer (NEB), 0.2 μM each of the outer primers (F3 and B3), 1.6 μM each of the inner primers (FIP and BIP), 0.8 μM each of the loop primers (LF and LB), 4 units of RNase inhibitor (Invitrogen), 7.5 units of WarmStart® RTx reverse transcriptase (NEB), 8 units of Bst 2.0 DNA polymerase (NEB), and 1-10 μL of sample or nuclease-free water (as negative control). RT-LAMP reactions were performed at 62° C. for 30 min.

In the experiments of real-time fluorescence detection of the LAMP products, 0.5×SYBR Green I dye (Invitrogen) was also added to the 25 μL of RT-LAMP reaction solution. Fluorescence signals were monitored and recorded every 1 min using a StepOnePlus™ Real-Time PCR System (Thermo Fisher Scientific).

Additional Information on CRISPR Cas12a-Mediated Fluorescence Detection

Sequences of the guide RNAs (gRNAs), recognizing the specific sequences of the RT-LAMP amplicons, are listed in Supporting Information Table 2. The ssDNA (8 nt) reporter was dually labeled with a fluorophore (6-carboxyfluorescein, 6-FAM) at the 5′ end and a quencher (Iowa Black® fluorescence quencher, IABk FQ) at the 3′ end. The ssDNA reporter served as a substrate for the trans-cleavage activity of Cas12a. For a few optimization experiments, a dsDNA activator was used, instead of the RT-LAMP amplicon. The dsDNA activator has the same sequence as the amplicon of the N gene. The gRNAs, the ssDNA reporter labeled with a fluorophore/quencher pair, and the dsDNA activator were all obtained from IDT (San Diego, Calif.)

Cas12a-mediated trans-cleavage of the ssDNA reporter, for the purpose of fluorescence detection, was carried out at room temperature (approximately 23° C.) for 10 min. The EnGen© Lba Cas12a enzyme (NEB) at 1 μM concentration was pre-incubated with 1.25 μM of gRNA in 1×NEBuffer 2.1 (NEB) to form the ribonucleoprotein (RNP) complex. An aliquot of the RNP complex solution was placed inside the cap of a PCR tube. The optimum concentrations of the Cas12a reaction reagents were 400 nM of the RNP complex, 40 mM MgSO₄, and 10 μM ssDNA reporter in 50 mM Tris-HCl buffer (pH=7.9).

Additional Information on Assay Kit for RT-LAMP and CRISPR Cas12a-Mediated Ambient Visualization in a Single Tube

The assay kit, consisting of a 0.2-mL PCR tube and a vial of rehydration buffer solution containing primers and the reporter, was convenient to be shipped to other locations for field applications. The PCR tube was prepared to contain dried (lyophilized) reagents for the assay. The mixture of the RT-LAMP reaction reagents, except the primers, was added at the bottom of the PCR tube. This mixture of the RT-LAMP reaction reagents was composed of 35 nmol of dNTP, 1×Isothermal Amplification Buffer, 150 nmol of MgSO₄, 4 units of RNase inhibitor, 7.5 units of WarmStart® RTx reverse transcriptase, 8 units of Bst 2.0 DNA polymerase and approximate 5 μmol of D-(+)-trehalose dihydrate (Sigma). The RNP complex (22 μmol) for the Cas12a-mediated reaction, 400 nmol of MgSO₄, and 500 nmol of Tris-HCl were added inside the cap of the tube. The tube was placed in a vacuum desiccator for 2 h to dry the reagents. After the reagents were dry, the tube was capped and stored at 4° C. until use. The vial of rehydration buffer contained the primers for RT-LAMP and 5 μM of ssDNA reporter for the Cas12a-mediated reaction. One vial of rehydration buffer contained the primer set for the N gene and the other vial of rehydration buffer contained the primer set for the E gene.

An aliquot (2-5 μL) of RNA extract sample and 20-23 μL buffer solution containing the primers and reporter were added into the bottom of the tube, mixing with and rehydrating the RT-LAMP reagents. The tube was capped and placed in a temperature controller and the bottom of the tube was maintained at 62° C. for 30 min. After 30 min of the RT-LAMP reactions, the tube was removed from the temperature controller, and left at the room temperature. Inverting and wrist-flicking of the tube mixed the RT-LAMP reaction product with the Cas12a reagents. After 10 min at the room temperature, green fluorescence was visualized under the excitation of a handheld UV lamp and a photo was recorded using a personal smartphone. The analysis of a RNA extract was complete in 40 min, including 30 min for RT-LAMP and 10 min for the Cas12a-mediated detection.

Tables

TABLE 1
Sequences of the amplified regions of the N gene
and E gene of SARS-CoV-2 as well as the primers
for the RT-LAMP reactions used in this study.
The complementary sequences are underlined.
The forward internal primer (FIP) consists
of an F1c region at the 5′ end and an F2
region at the 3′ end. The backward internal
primer (BIP) consists of a B1c region at the
5′ end and a B2 region at the 3′ end.

| | | SEQ |
|---|---|---|
| | | ID |
| Description | Sequences (5′-3′) | NO: |
| Amplified | AACACAAGCTTTCGGCAGACGTGGTCCAG | 1 |
| region | AACAAACCCAAGGAAATTTTGGGGACCAG | |
| of the N | GAACTAATCAGACAAGGAACTGATTACAA | |
| gene of | ACATTGGCCGCAAATTGCACAATTTGCCC | |
| SARS-CoV-2 | CCAGCGCTTCAGCGTTCTTCGGAATGTCG | |
| | CGCATTGGCATGGAAGTCACACCTTCGGG | |
| | AACGTGGTTGACCTACACAGGTGCCATCA | |
| | AATTGGATGACAAAGATCCAAATTTC | |
| N gene-F3 | AACACAAGCTTTCGGCAG | 2 |
| N gene-B3 | GAAATTTGGATCTTTGTCATCC | 3 |
| N gene-FTP | TGCGGCCAATGTTTGTAATCAG- | 4 |
| | CCAAGGAAATTTTGGGGAC | |
| N gene-BIP | CGCATTGGCATGGAAGTCAC- | 5 |
| | TTTGATGGCACCTGTGTAG | |
| N gene-LB | TTCCTTGTCTGATTAGTTC | 6 |
| N gene-LF | ACCTTCGGGAACGTGGTT | 7 |
| Amplified | CCGACGACGACTACTAGCGTGCCTTTGTA | 8 |
| region of | AGCACAAGCTGATGAGTACGAACTTATGT | |
| the E | ACTCATTCGTTTCGGAAGAGACAGGTACG | |
| gene of | TTAATAGTTAATAGCGTACTTCTTTTTCTT | |
| SARS-CoV-2 | GCTTTCGTGGTATTCTTGCTAGTTACACTA | |
| | GCCATCCTTACTGCGCTTCGATTGTGTGCG | |
| | TACTGCTGCAATATTGTTAACGTGAGTCTT | |
| | GTAAAACCTTCTTTTTACGTTTACTCT | |
| E gene-F3 | CCGACGACGACTACTAGC | 9 |
| E gene-B3 | AGAGTAAACGTAAAAAGAAGGTT | 10 |
| E gene-FTP | ACCTGTCTCTTCCGAAACGAA- | 11 |
| | TTTGTAAGCACAAGCTGATG | |
| E gene-BIP | CTAGCCATCCTTACTGCGCT- | 12 |
| | ACTCACGTTAACAATATTGCA | |
| E gene-LB | TGAGTACATAAGTTCGTAC | 13 |
| E gene-LF | TCGATTGTGTGCGTACTGC | 14 |

An RT-qPCR assay for the N2 target, according to the U.S. Center for Disease Control and Prevention (CDC) protocol, was used to compare and confirm the results from analyses using the RT-LAMP and CRISPR Cas12a-mediated assay. A 2-5 μL aliquot of RNA extract samples was mixed with the RT-qPCR master mix that was comprised of 1.5 μL of N2 primer-probe mix from the U.S. CDC EUA kit (IDT cat #10006606) and 1×TaqPath™ 1-Step RT-qPCR Master Mix (Thermo Fisher) to a final volume of 20 μL. The sequences of primers and probes are listed in Table 4. The reverse transcription and PCR thermal cycling parameters are summarized in Table 5, consistent with the U.S. CDC instruction of use (https://www.fda.gov/media/134922/download). An DNA standard, the N-gene plasmid purchased from IDT (IDT Cat #10006625), was used to calibrate and quantify synthetically transcribed RNA. The RNA samples with known copy numbers were used to generate standard curves for subsequent quantitation.

TABLE 2
Sequences of the RT-LAMP amplicons, gRNAs, DNA
activator, and ssDNA reporter labeled with a
fluorophore/quencher pair. The 20-nt spacer
sequences of the gRNAs, which recognize and
complement the target, are underlined.
The recognized region in the amplicon is
labeled in the matching color. The
protospacer adjacent motifs (PAMs) in
the N gene and E gene are labeled in bold.

| | | | SEQ |
|---|---|---|---|
| | | | ID |
| Description | Type | Sequences (5′-3′) | NO: |
| RT-LAMP | dsDNA | AATTGCACAATTTGCCCCCAGC | 15 |
| amplicon of | | GCTTCAGCGTTCTTCGGAATGT | |
| the N gene | | CG | |
| gRNA for the | RNA | UAAUUUCUACUAAGUGUAGAUCC | 16 |
| N gene | | CCCAGCGCUUCAGCGUUC | |
| Activator for | dsDNA | GCAAATTGCACAATTTGCCCCCA | 17 |
| the N gene | | GCGCTTCAGCGTTCTTCGGAATG | |
| | | TCGC | |
| RT-LAMP | dsDNA | ACGTTAATAGTTAATAGCGTACT | 18 |
| amplicon of | | TCTTTTTCTTGCTTTCGTGGTAT | |
| the E gene | | TCTTGCTAGTTACA | |
| gRNA for the | RNA | UAAUUUCUACUAAGUGUAGAUGU | 19 |
| E gene | | GGUAUUCUUGCUAGUUAC | |
| Reporter | ssDNA | /6-FAM/TTATTATT/IABk/ | |
| labeled with | | | |
| fluorophore | | | |
| and quencher | | | |

6FAM: 6carboxyfluorescein; IABk: Iowa Black® fluorescence quencher

TABLE 3
Results from the detection of SARS-CoV-2 in 100 human samples. The
analyses of the first 82 samples, from sample #1 to sample
#82, used 2 μL aliquots of each sample extract for the detection
of both the N gene and the E gene. The analyses of the last 18
samples, from sample #83 to sample #100, used 10 μL
of each sample extract for the detection of the N gene. There was
no sample remaining for the detection of the E gene in the last
18 samples (#83-#100). Typically, 5-10 samples were analyzed
in a batch; and each batch included a pair of negative control
and positive control (not all control images are shown). Both the
negative controls and the positive controls contained all reaction
reagents, except that nuclease-free water was used as the sample
in the negative control and 3750 copies of SARS-CoV-2 RNA was used
as the sample in the positive control. All the samples were analyzed
by a collaborating public health laboratory using the standard
RT-PCR method targeting the E gene. The Ct values from the RT-PCR
analyses of the E gene are included for information. “−”
indicates a negative fluorescence and “+” indicates a
positive strong green fluorescence.

| | Our assay | | Ct value of | |
|---|---|---|---|---|
| Samples | N-gene | E-gene | RT-PCR | Diagnosis |
| Negative control | − | − | N/A | Negative |
| Positive control | + | + | N/A | Positive |
| Sample #1 | − | − | Undetectable | Negative |
| Sample #2 | + | + | 23.0 | Positive |
| Sample #3 | + | + | 28.1 | Positive |
| Sample #4 | − | − | Undetectable | Negative |
| Sample #5 | − | − | 33.0 | False Negative |
| Sample #6 | + | + | 28.9 | Positive |
| Sample #7 | − | − | Undetectable | Negative |
| Sample #8 | − | − | Undetectable | Negative |
| Sample #9 | + | + | 11.7 | Positive |
| Sample #10 | + | + | 19.6 | Positive |
| Sample #11 | + | + | 17.8 | Positive |
| Sample #12 | − | − | Undetectable | Negative |
| Sample #13 | − | − | Undetectable | Negative |
| Sample #14 | − | − | Undetectable | Negative |
| Sample #15 | + | + | 19.6 | Positive |
| Sample #16 | + | + | 19.0 | Positive |
| Sample #17 | − | − | Undetectable | Negative |
| Sample #18 | − | − | Undetectable | Negative |
| Sample #19 | − | − | Undetectable | Negative |
| Sample #20 | + | + | 24.5 | Positive |
| Sample #21 | + | + | 24.3 | Positive |
| Sample #22 | − | − | Undetectable | Negative |
| Sample #23 | − | − | Undetectable | Negative |
| Sample #24 | + | + | 30.3 | Positive |
| Sample #25 | + | + | 23.7 | Positive |
| Sample #26 | − | − | Undetectable | Negative |
| Sample #27 | − | − | Undetectable | Negative |
| Sample #28 | + | + | 25.9 | Positive |
| Sample #29 | − | − | Undetectable | Negative |
| Sample #30 | − | − | Undetectable | Negative |
| Sample #31 | − | − | Undetectable | Negative |
| Sample #32 | + | + | 26.6 | Positive |
| Sample #33 | + | + | 28.3 | Positive |
| Sample #34 | − | − | Undetectable | Negative |
| Sample #35 | − | − | Undetectable | Negative |
| Sample #36 | − | − | Undetectable | Negative |
| Sample #37 | + | + | 27.2 | Positive |
| Sample #38 | + | + | 28.2 | Positive |
| Sample #39 | − | − | Undetectable | Negative |
| Sample #40 | − | − | Undetectable | Negative |
| Sample #41 | − | − | Undetectable | Negative |
| Sample #42 | − | − | Undetectable | Negative |
| Sample #43 | + | + | 23.6 | Positive |
| Sample #44 | − | − | Undetectable | Negative |
| Sample #45 | − | − | Undetectable | Negative |
| Sample #46 | − | − | Undetectable | Negative |
| Sample #47 | − | − | Undetectable | Negative |
| Sample #48 | + | + | 23.5 | Positive |
| Sample #49 | − | − | Undetectable | Negative |
| Sample #50 | − | − | Undetectable | Negative |
| Sample #51 | − | − | Undetectable | Negative |
| Sample #52 | − | − | Undetectable | Negative |
| Sample #53 | − | − | Undetectable | Negative |
| Sample #54 | − | − | Undetectable | Negative |
| Sample #55 | − | − | Undetectable | Negative |
| Sample #56 | − | − | Undetectable | Negative |
| Sample #57 | − | − | Undetectable | Negative |
| Sample #58 | − | − | Undetectable | Negative |
| Sample #59 | − | − | Undetectable | Negative |
| Sample #60 | − | − | Undetectable | Negative |
| Sample #61 | − | − | Undetectable | Negative |
| Sample #62 | − | − | Undetectable | Negative |
| Sample #63 | + | + | 23.4 | Positive |
| Sample #64 | − | − | Undetectable | Negative |
| Sample #65 | − | − | Undetectable | Negative |
| Sample #66 | − | − | Undetectable | Negative |
| Sample #67 | − | − | Undetectable | Negative |
| Sample #68 | − | − | Undetectable | Negative |
| Sample #69 | − | − | Undetectable | Negative |
| Sample #70 | − | − | Undetectable | Negative |
| Sample #71 | − | − | Undetectable | Negative |
| Sample #72 | + | + | 22.4 | Positive |
| Sample #73 | + | + | 21.2 | Positive |
| Sample #74 | + | + | 22.2 | Positive |
| Sample #75 | + | + | 23.1 | Positive |
| Sample #76 | + | + | 24.0 | Positive |
| Sample #77 | + | + | 25.0 | Positive |
| Sample #78 | + | + | 19.6 | Positive |
| Sample #79 | + | + | 26.1 | Positive |
| Sample #80 | + | + | 27.0 | Positive |
| Sample #81 | + | + | 29.1 | Positive |
| Sample #82 | + | + | 28.2 | Positive |
| Sample #83 | + | N/A | 31.9 | Positive |
| Sample #84 | + | N/A | 33.1 | Positive |
| Sample #85 | + | N/A | 30.9 | Positive |
| Sample #86 | + | N/A | 35.0 | Positive |
| Sample #87 | + | N/A | 33.3 | Positive |
| Sample #88 | + | N/A | 34.1 | Positive |
| Sample #89 | + | N/A | 31.1 | Positive |
| Sample #4 | − | N/A | Undetectable | Negative |
| Sample #90 | + | N/A | 30.6 | Positive |
| Sample #91 | − | N/A | 39.2 | False Negative |
| Sample #92 | + | N/A | 30.8 | Positive |
| Sample #93 | + | N/A | 30.9 | Positive |
| Sample #94 | + | N/A | 33.6 | Positive |
| Sample #95 | − | N/A | 33.6 | False Negative |
| Sample #96 | + | N/A | 33.7 | Positive |
| Sample #97 | + | N/A | 31.1 | Positive |
| Sample #98 | + | N/A | 30.2 | Positive |
| Sample #99 | + | N/A | 32.6 | Positive |
| Sample #100 | + | N/A | 32.9 | Positive |

N/A: not available.

TABLE 4
Sequences of the primers and the probes
for the RT-qPCR assay.

| Description | Sequences (5′-3′) |
|---|---|
| Amplified | TTACAAACATTGGCCGCAAATTGCACAATTTG |
| fragments of | CCCCCAGCGCTTCAGCGTTCTTCGGAATGTCG |
| SARS-CoV-2 (N2) | CGC (SEQ IN NO: 20) |
| N2-Forward | TTACAAACATTGGCCGCAAA |
| primer | (SEQ IN NO: 21) |
| N2-Reverse | GCGCGACATTCCGAAGAA (SEQ IN |
| primer | NO: 22) |
| N2-Probe | 6-FAM/ACAATTTGCCCCCAGCGCTTCAG/ |
| | BHQ_1 (SEQ IN NO: 23) |

6-FAM: 6-carboxyfluorescein; BHQ_1: Black Hole Quencher®-1

TABLE 5
Thermal cycling conditions for RT-qPCR conducted
in parallel with the RT-LAMP CRISPR-Cas12a assay.

| Steps | Temperature | Duration | | Cycles |
|---|---|---|---|---|
| UNG activation | 25° C. | 2 | min | N/A |
| Reverse Transcription | 50° C. | 15 | min | |
| Polymerase activation | 95° C. | 2 | min | |
| Denaturation | 95° C. | 3 | sec | 50 |
| Extension and detection | 60° C. | 30 | sec | |

TABLE 6
Summary of results obtained from the detection of the N gene
of SARS-CoV-2 at very low copy numbers. The experiment was
repeated in four batches. In each batch, samples containing
different concentrations of the viral RNA (0, 1, 5, 10, 15 and
30 copies/μL) were analyzed in triplicate. Five microliters
(5 μL) of sample was used for each reaction.

| | | | 5 | 10 | 15 | 30 |
|---|---|---|---|---|---|---|
| Con- | No | 1 | copies/ | copies/ | copies/ | copies/ |
| centration | target | copy/μL | μL | μL | μL | μL |
| # of tests | 12 | 12 | 12 | 12 | 12 | 12 |
| # of positives | 0 | 3 | 3 | 8 | 10 | 12 |
| Positive rate | N/A | 25% | 25% | 67% | 83% | 100% |

TABLE 7
Summary of results obtained from RT-qPCR detection of the N
gene of SARS-CoV-2 at very low copy numbers. The experiment was
repeated in five batches. In each batch, samples containing different
concentrations of the viral RNA (0, 1, 2, 5, 10, 15 and 30 copies/μL)
were analyzed in triplicate. Five microliters (5 μL) of sample was used
for each reaction.

| | No | 1 copy/ | 2 copies/ | 5 copies/ | 10 copies/ | 15 copies/ | 30 copies/ |
|---|---|---|---|---|---|---|---|
| Concentration | target | μL | μL | μL | μL | μL | μL |
| # of tests | 15 | 15 | 15 | 15 | 15 | 15 | 15 |
| # of positives | 0 | 5 | 7 | 14 | 15 | 14 | 15 |
| Average Ct | N/A | 36.0 ± 1.1 | 35.6 ± 0.6 | 35.1 ± 1.0 | 33.7 ± 0.9 | 33.1 ± 0.4 | 32.3 ± 0.6 |
| value | | | | | | | |
| Positive rate | N/A | 33% | 47% | 93% | 100% | 93% | 100% |

The embodiments described herein are intended to be examples only. Alterations, modifications and variations can be effected to the particular embodiments by those of skill in the art. The scope of the claims should not be limited by the particular embodiments set forth herein, but should be construed in a manner consistent with the specification as a whole.

All publications, patents and patent applications mentioned in this Specification are indicative of the level of skill those skilled in the art to which this invention pertains and are herein incorporated by reference to the same extent as if each individual publication patent, or patent application was specifically and individually indicated to be incorporated by reference.

The invention being thus described, it will be obvious that the same may be varied in many ways. Such variations are not to be regarded as a departure from the spirit and scope of the invention, and all such modification as would be obvious to one skilled in the art are intended to be included within the scope of the following claims.

1. A method of detecting SARS-CoV-2 in a sample from a subject having a SARS-CoV-2 infection, or suspected of having a SARS-CoV-2 infection, or at risk of a SARS-CoV-2 infection, comprising:
(a) subjecting the sample to reverse transcription loop-mediated exponential amplification (RT-LAMP) to amplify a target sequence with said SARS-CoV-2 and generate an RT-LAMP amplicon comprising the target sequence;
(b) contacting the RT-amplicon with a reaction mixture at a reaction temperature for a reaction time, the reaction mixture comprising:
a guide RNA (gRNA) comprising a polynucleotide sequence complementary to the target sequence recognizing,
a Cas12a protein; and
a ssDNA reporter molecules comprising a first end having a quencher molecule and a second end having a fluorophore
(c) detecting a signal indicative of amplification of the region of the target nucleic acid molecule,
wherein, step (a) and step (b) are performed sequentially in a single unopened container,
wherein, if a signal detected, the subject is determined to have a SARS CoV-2 infection,
wherein, if a signal is not detected, the subject is determined to not to have a SARS CoV-2 infection.

2. The method of claim 1, wherein the target sequence is the E gene or N gene.

3. The method of claim 1, wherein the gRNA comprises or consists of 41 nucleotides.

4. The method of claim 3, wherein the gRNA comprises or consists of the nucleotide sequence of SEQ ID NO: 16 or SEQ ID NO: 19.

5. The method of claim 1, wherein the ssDNA reporter comprises or consists of 8 nucleotides.

6. The method of claim 5, wherein the ssDNA reporter comprises or consists of the nucleotide sequence 6-FAM/TTATTATT/IABk.

7. The method of claim 5, wherein the quencher molecule is Iowa Black® fluorescence quencher and the fluorophore is 6-carboxyfluorescein.

8. The method of claim 1, wherein said reverse transcription loop-mediated exponential amplification (RT-LAMP) is carried out at about 62° C. for about 30 min.

9. The method of claim 1, wherein the reaction time in step (b) is about 10 minutes and the reaction temperature is about 23° C.

10. The method of claim 1, wherein the reagents for step (a) and step (b) are lyophilized and present in separate compartments of the single container, before addition of the sample.

11. The method of claim 1, wherein said subject is a human.

12. An isolated polynucleotide comprising or consisting of the nucleotide sequence of SEQ ID Nos. 1, 2, 3, 4, 5, 6, 7, 8, 9, 10, 11, 12, 13, 14, 15, 16, 17, 18, 19, 20, 21, 22, or 23.

13. A kit comprising one or more isolated polynucleotides comprising or consisting of the nucleotide sequence of SEQ ID Nos. 1, 2, 3, 4, 5, 6, 7, 8, 9, 10, 11, 12, 13, 14, 15, 16, 17, 18, 19, 20, 21, 22, or 23, and optionally a container, and/or optionally instructions for the use thereof.
